(19) United States
(12) Patent Application Publication
(10) Pub. No.: US 2006/0204556 A1
(43) Pub. Date: Sep. 14, 2006

(54) Cell-loaded prostheses for regenerative intraluminal applications

(75) Inventors: Eric Daniels, Encinitas, CA (US); Brian M. Strem, San Diego, CA (US); Susan Lynn Riley, San Diego, CA (US); Mike De Emedio, San Diego, CA (US); John K. Fraser, San Diego, CA (US); Marc H. Hedrick, Encinitas, CA (US)

Correspondence Address: Kenton R. Mullins;Stout, Uxa, Buyan & Mullins, LLP, Suite 300, 4 Venture, Irvine, CA, 92618, US

(73) Assignee: Cytori Therapeutics, Inc., San Diego, CA (US)

(21) Appl. No.: 11/317,422

(22) Filed: Dec. 22, 2005

Related U.S. Application Data
(63) Continuation-in-part of application No. 10/885,293, filed on Jul. 1, 2004.
(63) Continuation-in-part of application No. 10/316,127, filed on Dec. 9, 2002.
(60) Provisional application No. 60/338,856, filed on Dec. 7, 2001.
(60) Provisional application No. 60/554,455, filed on Mar. 19, 2004.

(51) Int. Cl. A61K 35/12; A61F 2/06; A61K 9/70

(52) U.S. Cl. 424/443; 424/93.7; 623/1.11

(57) ABSTRACT

Cell-loaded devices or prostheses having various applications such as insertion into body passages are disclosed. The prostheses include cell carrier portions which are compatible with living tissue and which are loaded with therapeutic cell populations, and the prostheses can be applied within or replace one or more of narrow segments, environments which may be difficult to access or luminal areas of the body such as parts of blood vessels. In the context of blood vessels, the cell-loaded devices or prostheses can line or otherwise treat with therapeutic cell populations inner walls of damaged blood vessels and surrounding parenchyma or other organs.

### CROSS-REFERENCE TO RELATED APPLICATIONS

This application claims the benefit of U.S. Provisional Application No. 60/639,013, filed Dec. 22, 2004. This application is a continuation-in-part application of U.S. application Ser. No. 10/885,293, filed Jul. 1, 2004 and entitled CELL CARRIER AND CELL CARRIER CONTAINMENT DEVICES CONTAINING REGENERATIVE CELLS, which application is a continuation-in-part of U.S. application Ser. No. 10/316,127, filed on Dec. 9, 2002 and entitled SYSTEMS AND METHODS FOR TREATING PATIENTS WITH PROCESSED LIPOASPIRATE CELLS, which claims the benefit of U.S. Provisional Application No. 60/338,856, filed Dec. 7, 2001, and which application also claims the benefit of U.S. Provisional Application No. 60/554,455, filed Mar. 19, 2004. The entire contents of the aforementioned applications are expressly incorporated herein by this reference.

### BACKGROUND OF THE INVENTION

1. Field of the Invention

The present invention relates generally to medical devices and, more particularly, stents.

2. Description of Related Art

In recent years a number of minimally invasive techniques have been developed to treat occlusive vascular disease, and to repair aneurysms occurring in the circulatory system, vessels, and other organs. In occlusive vascular disease, such as arteriosclerosis, plaque accumulates within a vessel and gradually narrows the vessel to a degree that the vessel can no longer supply an adequate flow of blood. A number of vascular prostheses have been developed to re-expand and retain the patency of such afflicted vessels, for example, after atherectomy or angioplasty. In the coronary artery both bare and drug eluting stents are currently used to treat blockages, with the drug eluting component being used to treat in-stent restenosis.

### SUMMARY OF THE INVENTION

Cell-loaded prostheses having various applications are disclosed which comprise cell carrier portions loaded with therapeutic cell populations and which can be applied within or replace parts of for example blood vessels or other luminal areas (e.g., hepatobiliary) of the body of a living animal or a living human or be placed in other environments which may be for example difficult to access. In the context of regenerative intraluminal applications, the cell-loaded prostheses may include flexible tubular bodies, the diameters of which can be decreased or increased. The invention may be particularly useful, for example, for mechanical intraluminal or transluminal implantation and may take the form of an expanded self-fixating cell-loaded prosthesis (e.g., stent) for blood vessels, hepatobiliary, gastrointestinal, and respiratory tracts or the like. By means of a cell-loaded device or prosthesis of the present invention, the inner walls of damaged blood vessels and surrounding parenchyma (e.g., cardiomyocytes) or other organs may be lined and/or otherwise treated with therapeutic cell populations.

In the context of stents, the present application has as an objective to move beyond in-stent restenosis to provide the patient a stent, drug-eluting or not, comprising (e.g., coated with) a cell carrier in the form of a bioresorbable polymer which may in turn be loaded (e.g., in real-time) with a therapeutic cell population. The present application may have as another objective to provide the patient a stent, drug-eluting or not, comprising (e.g., coated with) a bioresorbable polymer which may in turn be loaded with a therapeutic cell population using cell culture and uncultured cells (e.g., real-time therapy) from a variety of tissue sources. The polymer can be formed into or onto the stent during or following a manufacturing process of the stent, using techniques known to those skilled in the art. In embodiments wherein the polymer is formed (e.g., applied) onto the stent during the manufacturing process of the stent, known coating or dipping methods may be implemented.

The cell-loaded prostheses of the present invention may be applicable to, for example, surgical and other medicinal techniques, where there may be a need for inserting and/or expanding a cell-loaded device in for example blood vessels, urinary tracts or other places that are difficult to access which has for its function to treat with therapeutic cell populations and/or support the vessel or tract and which can be left in an implanted position.

The cell-loaded prostheses according to the present invention can be used also in many medicinal applications, including utilization in different types of aneurysm reflected by some form of vessel widening and outpouching, or the opposite, stenosis, involving constriction of blood vessels. In the context of such mechanical-type applications, the invention can be used for example to support and keep open vessels of vascular systems, to close pathological vessel abnormalities, to bridge pathological vessel dilatations and ruptures in interior vessel walls or for example to stabilize bronchial tubes and bronchi. The cell-loaded device according to the present invention may also be designed to act as a filter for thrombosis, for example by application in Vena Cava Inferior to prevent the formation of lung emboliae. Other applications may include treatment with therapeutic cell populations, alone or in combination with any of the above mechanical-type applications. The invention may be particularly suited to be used as a cell-loaded prosthesis, for example a graft, for application in blood vessels or other tubular organs within the body.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description.

### BRIEF DESCRIPTION OF THE DRAWINGS

FIG. 1A and FIG. 1B show diagrammatically a side view and an end view, respectively, of the flexible tubular body according to the invention;

FIG. 2A and FIG. 2B show the same tubular body as in FIG. 1 but in contracted state;

FIG. 3 and FIG. 4 show one separate thread member of the body, the body being shown in contracted and expanded state, respectively;

FIG. 5 shows diagrammatically an assembly incorporating the tubular body according to the present invention;

FIG. 6 shows in an enlarged view part of the assembly of FIG. 5;

FIG. 7 shows an alternative embodiment of the tubular body;

FIG. 8 shows the tubular body designed as a combined graft and filter;

FIG. 9 shows the tubular body used as a graft in connection to aneurism;

FIG. 10 shows a diagram of the diameter (D) of the body as a function of the angle alpha and of the elongation of the cell-loaded prosthesis in %;

FIG. 11 shows diagrammatically an alternative assembly for manipulating the cell-loaded prosthesis of the invention;

FIG. 12 is a side elevation, partly in section, of an instrument according to the invention;

FIG. 13 is an enlarged side elevation of the distal end of an instrument showing a partly deployed stent:

FIG. 14 is an enlarged cross section of one embodiment of a grip member;

FIG. 15 is a cross-section through line 15A-15A on FIG. 14.

FIG. 16 and FIG. 17 are cross-sections of alternative grip members; and

FIG. 18 is a side elevation of part of an instrument according to the invention showing other features.

FIG. 19 is a side elevation of a self-expanding stent constructed according to the present invention;

FIG. 20 is an end elevational view of the stent;

FIG. 21 is an enlarged partial view of one of the composite filaments forming the stent;

FIG. 22 is an enlarged sectional view taken along the line 4-4 in FIG. 3;

FIGS. 23-27 schematically illustrate a process for manufacturing the stent;

FIG. 28 schematically illustrates a swaging step of an alternative process for manufacturing the stent;

FIG. 29 is an end elevational view of an alternative embodiment filament;

FIG. 30 is an elevational view of several components of an alternative composite filament constructed according to the present invention;

FIG. 31 is an end elevational view of the composite filament formed by the components shown in FIG. 12;

FIG. 32 is an end elevational view of another alternative embodiment composite filament;

FIG. 33 illustrate a stent or cell-loaded prosthesis in accordance with one embodiment of the present invention.

FIG. 34 depicts a balloon catheter with a stent attached in its contracted position;

FIG. 35 shows the balloon catheter in an expanding state, while heat is applied through the heating source, and the corresponding expansion of the stent

FIG. 36 depicts the fully expanded balloon catheter and the maximally expanded stent; and

FIG. 37 depicts the deflated balloon catheter as it is being withdrawn from the body cavity, with the stent remaining in place.

### DETAILED DESCRIPTION OF THE PRESENTLY PREFERRED EMBODIMENTS

In accordance with the present invention, one illustrative embodiment provides a combination of a cell-loaded prosthesis having a therapeutically effective dose of regenerative cells, e.g., adipose-derived adult stem and/or progenitor cells, that may promote one or more of a) repopulation of the endothelial layer which may be denuded upon insertion of the cell-loaded prosthesis, e.g., stent; b) action on surrounding myocardium for promotion of collateralogenesis or formation of collateral vessels; and c) cardiomyocytic differentiation. The cell-loaded prosthesis of the present invention may thus be useful for treating disorders requiring, for example, vessel or cardio formation or treatment.

The present invention also relates to rapid and reliable systems and methods for separating and concentrating regenerative cells, e.g., stem cells and/or progenitor cells, from a wide variety of tissues, including but not limited to, adipose, bone marrow, blood, skin, muscle, liver, connective tissue, fascia, brain and other nervous system tissues, blood vessels, and other soft or liquid tissues or tissue components or tissue mixtures (e.g., a mixture of tissues including skin, blood vessels, adipose, and connective tissue), and to the integration of those regenerative cells onto prosthesis for treatments including regenerative intraluminal, transluminal and related applications.

In order that the present invention may be more readily understood, certain terms are first defined. Additional terms and definitions may be set forth throughout the detailed description.

As used herein, “regenerative cells” refers to any heterogeneous or homologous cells obtained using the systems and methods of the present invention which cause or contribute to complete or partial regeneration, restoration, or substitution of structure or function of an organ, tissue, or physiologic unit or system to thereby provide a therapeutic, structural or cosmetic benefit. Examples of regenerative cells include: adipose derived cells (ASCs), endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

One mechanism by which the regenerative cells may provide a therapeutic, structural or cosmetic benefit can be by incorporating themselves or their progeny into newly generated, existing or repaired tissues or tissue components. For example, ASCs and/or their progeny may incorporate into newly generated bone, muscle, or other structural or functional tissue and thereby cause or contribute to a therapeutic, structural or cosmetic improvement. Similarly, endothelial cells or endothelial precursor or progenitor cells and their progeny may incorporate into existing, newly generated, repaired, or expanded blood vessels to thereby cause or contribute to a therapeutic, structural or cosmetic benefit. Another mechanism by which the regenerative cells may provide a therapeutic, structural or cosmetic benefit is by expressing and/or secreting molecules, e.g., growth factors, that promote creation, retention, restoration, and/or regeneration of structure or function of a given tissue or tissue component.

The regenerative cells may be used in their ‘native’ form as present in or separated and concentrated from the tissue using the systems and methods of the present invention or they may be modified by stimulation or priming with growth factors or other biologic response modifiers, by gene transfer (transient or stable transfer), by further sub-fractionation of the resultant population on the basis or physical properties (for example size or density), differential adherence to a solid phase material, expression of cell surface or intracellular molecules, cell culture or other ex vivo or in vivo manipulation, modification, or fractionation as further described herein. The regenerative cells may also be used in combination with other cells or devices such as synthetic or biologic prostheses (e.g., stents), scaffolds, materials or devices that deliver factors, drugs, chemicals or other agents that modify or enhance the relevant characteristics of the cells as further described herein.

As used herein, “regenerative cell composition” refers to the composition of cells typically present in a volume of liquid after a tissue, e.g., adipose tissue, is washed and at least partially disaggregated. For example, a regenerative cell composition of the invention comprises multiple different types of regenerative cells, including ASCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes. The regenerative cell composition may also contain one or more contaminants, such as collagen, which may be present in the tissue fragments, or residual collagenase or other enzyme or agent employed in or resulting from the tissue disaggregation process described herein.

As used herein, “regenerative medicine” refers to any therapeutic, structural or cosmetic benefit that can be derived from the placement, either directly or indirectly, of regenerative cells into a subject.

As used herein, “stem cell” refers to a multipotent regenerative cell with the potential to differentiate into a variety of other cell types, which perform one or more specific functions and have the ability to self-renew. Some of the stem cells disclosed herein may be multipotent.

As used herein, “progenitor cell” refers to a multipotent regenerative cell with the potential to differentiate into more than one cell type and has limited or no ability to self-renew. “Progenitor cell,” as used herein, also refers to a unipotent cell with the potential to differentiate into only a single cell type, which performs one or more specific functions and has limited or no ability to self-renew. In particular, as used herein, “endothelial progenitor cell” refers to a multipotent or unipotent cell with the potential to differentiate into vascular endothelial cells.

As used herein, “precursor cell” refers to a unipotent regenerative cell with the potential to differentiate into one cell type. Precursor cells and their progeny may retain extensive proliferative capacity, e.g., lymphocytes and endothelial cells, which can proliferate under appropriate conditions.

As used herein “stem cell number” or “stem cell frequency” refers to the number of colonies observed in a clonogenic assay in which ADCs are plated at low cell density (<10,000 cells/well) and grown in growth medium supporting MSC growth (for example, DMEM/F12 medium supplemented with 10% fetal calf serum, 5% horse serum, and antibiotic/antimycotic agents). Cells are grown for two weeks after which cultures are stained with hematoxylin and colonies of more than 50 cells are counted as CFU-F. Stem cell frequency can be calculated as the number of CFU-F observed per 100 nucleated cells plated (for example; 15 colonies counted in a plate initiated with 1,000 nucleated regenerative cells gives a stem cell frequency of 1.5%). Stem cell number can be calculated as stem cell frequency multiplied by the total number of nucleated ADC cells obtained. A high percentage (˜100%) of CFU-F grown from regenerative cells express the cell surface molecule CD105 which is also expressed by marrow-derived stem cells (Barry et al., 1999). CD105 is also expressed by adipose tissue-derived stem cells (Zuk et al., 2002).

As used herein, the term “adipose tissue” refers to fat including the connective tissue that stores fat. Adipose tissue contains multiple regenerative cell types, including ASCs and endothelial progenitor and precursor cells.

As used herein, the term “unit of adipose tissue” refers to a discrete or measurable amount of adipose tissue. A unit of adipose tissue may be measured by determining the weight and/or volume of the unit. Based on the data identified above, a unit of processed lipoaspirate, as removed from a patient, has a cellular component in which at least 0.1% of the cellular component is stem cells; that is, it has a stem cell frequency, determined as described above, of at least 0.1%. In reference to the disclosure herein, a unit of adipose tissue may refer to the entire amount of adipose tissue removed from a patient, or an amount that is less than the entire amount of adipose tissue removed from a patient. Thus, a unit of adipose tissue may be combined with another unit of adipose tissue to form a unit of adipose tissue that has a weight or volume that is the sum of the individual units.

As used herein, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a patient is a portion of the removed adipose tissue.

As used herein, the term “processed lipoaspirate” refers to adipose tissue that has been processed to separate the active cellular component (e.g., the component containing regenerative) from the mature adipocytes and connective tissue. This fraction is referred to herein as “adipose derived cells” or “ADC.” Typically, ADC refers to the pellet of regenerative cells obtained by washing and separating and concentrating the cells from the adipose tissue. The pellet is typically obtained by centrifuging a suspension of cells so that the cells aggregate at the bottom of a centrifuge chamber or cell concentrator.

As used herein, the terms “administering,” “introducing,” “delivering,” “placement” and “transplanting” are used interchangeably herein and refer to the placement of the regenerative cells of the invention into a subject by a method or route which results in at least partial localization of the regenerative cells at a desired site. The regenerative cells can be administered by any appropriate route which results in delivery to a desired location in the subject where at least a portion of the cells or components of the cells remain viable. The period of viability of the cells after administration to a subject can be as short as a few hours, e.g., twenty-four hours, to a few days, to as long as several years.

As used herein, the term “treating” includes reducing or alleviating at least one adverse effect or symptom of a disease or disorder

As used herein, “therapeutically effective dose of regenerative cells” refers to an amount of regenerative cells that are sufficient to bring about a beneficial or desired clinical effect. Said dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, the term “subject” includes warm-blooded animals, preferably mammals, including humans. In a preferred embodiment, the subject can be a primate. In an even more preferred embodiment, the subject can be a human.

Reference will now be made in detail to the presently preferred embodiments of the invention, examples of which are illustrated in the accompanying figures. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention. The present invention may be practiced in conjunction with various techniques that are conventionally used in the art, and only so much of the commonly practiced structures and steps are included herein as are necessary to provide an understanding of the present invention.

According to the present invention, cell-loaded prostheses are provided comprising cell carriers (e.g., polymers), which in turn may be loaded with therapeutic cell populations (e.g., components including regenerative cells). In one implementation, cell-loaded stents are provided which may or may not be drug-eluting, and which may comprise or be coated with bioresorbable polymers that are loaded in real-time with therapeutic cell populations.

The polymer can be formed into or onto a stent during or following a manufacturing process of the stent, using techniques known to those skilled in the art. In embodiments wherein the polymer is formed (e.g., applied) onto the stent during the manufacturing process of the stent, known coating or dipping methods may be implemented. As examples, one or more of the following polymers can be used: 70/30 L:DL, 85/15 DL:G, 85/15 L:G, 50/50 DL:G, 70/30 L:CL, 70/30 L:TMC, and combinations thereof.

In implementations wherein the polymer is pre-coated onto the stent, the cell population to be used can be loaded on the polymer-coated stent, using, for example, one or more of a dynamic and a static seeding device. The cell population can include, but is not limited to, ADC, bone-marrow derived cells, and peripheral blood derived cells, wherein the cells may consist of or comprise, for example, regenerative cells such as stem cells. The stem cells may be embryonic or adult stem cells, and parts or all of the cell population may be cultured or non-cultured. Once the polymer is seeded with the population or multiple subpopulations of cells, the stent can be inserted into a body passage such as the coronary artery.

Mechanisms of action of the implantation of a cell-loaded prosthesis of the present invention can be multiple. The cell populations found on the polymer can have several modes of action. First, certain ones of the cell populations can be formed to contain endothelial progenitor cells (EPCs) or the polymers can be chosen for their ability to select this particular subpopulation of cells from a mixture of cells present. Such endothelial progenitor cells may act locally to repopulate the endothelial layer which may be denuded upon insertion of the stent. Secondarily, components (e.g., cells), such as these endothelial progenitor cells, can act locally in the surrounding myocardium to promote collateralogenesis or formation of new collateral vessels. In addition to the angiogenic properties of the endothelial progenitor cells, stem cells from, for example, adipose tissue or marrow may be implemented to effectuate cardiomyocytic differentiation, wherein such differentiation may in some implementations be responsive to ischemic injury or part of a larger inflammatory environment. Thirdly, certain ones of the cell population are known to produce growth factors, small molecules, and cytokines (e.g., HGF, PLGF) which may act to convert stunned or hibernating myocardium or act to stimulate endogenous cardiac precursor cells. This local niche manipulation may have a beneficial effect on global cardiac function. As such, the cell-loaded stents of the present invention can serve as vectors for the delivery of precursor cells.

1. Obtaining Regenerative Cells (ADC)

As previously set forth herein, regenerative cells, e.g., stem and progenitor cells, can be harvested from a wide variety of tissues. The system of the present invention may be used for all such tissues. Adipose tissue, however, is an especially rich source of regenerative cells. The methods, structure and discussion in connection with obtaining regenerative cells as disclosed in the corresponding section of U.S. application Ser. No. 10/885,293 are incorporated herein by reference, in any combination, in whole or in part, with any modification as may be apparent or recognizable to one skilled in the art, to the extent compatible and not mutually exclusive.

2. Cell Carrier Portion of the Cell-Loaded Prosthesis

As mentioned, in accordance with embodiments of the present invention, cell-loaded prostheses are provided comprising cell carrier portions (e.g., bioresorbable polymers), which in turn can be loaded with therapeutic cell populations (e.g., components including regenerative cells). The cell carrier portions of the cell-loaded prostheses can in certain embodiments be important or critical for maintaining or facilitating regenerative or treatment functions of the regenerative cells. Specifically, the cell carrier, on which the regenerative cells may be placed, as further described herein, can serve to organize the cells in three dimensions. Accordingly, in considering substrate materials, ones that exhibit clinically acceptable biocompatibility may be chosen. In addition, the mechanical properties of the cell carrier may be formed to be sufficient so that it does not collapse or otherwise unduly lose integrity or desired functionality over the passage of time and/or during for example the patient's normal activities.

A variety of cell carriers, known and used in the art, are intended to be encompassed and applicable for use (e.g., formation with, on, or within) the cell-loaded prostheses of the present invention. Cell carriers in the form of gels, such as hydrogels, are encompassed by the present invention. Fiber based cell carriers are also encompassed by the present invention and include woven meshes, knitted meshes, non-woven meshes (felts) and polymer coated meshes (e.g., to enhance strength). Methods for manufacturing fiber based cell carriers are known in the art. For example, cell carriers based on knitted meshes can be made according to the methods described by H. J. Buchsbaum, W. Christopherson, S. Lifshitz, and S. Bernstein. Vicryl mesh in pelvic floor reconstruction. Arch. Surg 120 (12):1389-1391, 1985. Non-woven mesh based cell carriers can be made according to the methods described by L. E. Freed, G. Vunjak-Novakovic, R. J. Biron, D. B. Eagles, D. C. Lesnoy, S. K. Barlow, and R. Langer. Biodegradable polymer cell carriers for tissue engineering. Biotechnology (NY) 12 (7):689-693, 1994. Polymer coated meshes (e.g., to enhance strength) can be made according to the methods described by W. S. Kim, J. P. Vacanti, L. Cima, D. Mooney, J. Upton, W. C. Puelacher, and C. A. Vacanti. Cartilage engineered in predetermined shapes employing cell transplantation on synthetic biodegradable polymers. Plast Reconstr Surg 94 (2):233-237, 1994.

In one embodiment, the cell carrier portion of the cell-loaded prosthesis is preformed into particulates. The sizes and shapes of these particulates can range from, for example, less than 1 mm to up to 10 mm in diameter and from abstract shaped granules to defined shapes, respectively. These particulates can either be highly porous (e.g., greater than 95% void volume) or non-porous. The cell carrier particulates can be used, for example, to contain the cells into the center of or around the outside of the cell-loaded prosthesis and/or can be preloaded into or onto the cell-loaded prosthesis before packaging and sterilization or can be packaged and sterilized separately from the cell-loaded prosthesis.

Sponge or foam based cell carriers are also encompassed by the present invention and can be manufactured using one or more methods such as solvent casted-particulate leached (SC-PL), melt molded-particulate leached (where melt molding can include e.g., compression molding, injection molding or extrusion), extrusion-particulate leaching, emulsion-freeze drying, freeze drying combined with particulate-leaching, solution casting, gel casting, atomized foams, phase separation, phase separation combined with particulate leaching, high pressure CO2, gas foaming of effervescent salts, 3D printing, fused deposition modeling and methods using electrospun nanofibers or other nanofibers. In one embodiment, the cell carrier portion of the present invention is manufactured using known coating or dipping methods, and in another embodiment it is manufactured using at least in part solvent casting/freeze drying methods.

Methods for manufacturing a sponge or foam based cell carrier of the invention are known in the art. For example, solvent casted-particulate leached (SC-PL) cell carriers can be made according to the methods disclosed in A. G. Mikos, A. J. Thorsen, L. A. Czerwonka, Bao Y., and R. Langer. Preparation and characterization of poly(L-lactic acid) foams. Polymer 35 (5):1068-1077, 1994 and P. X. Ma and J. W. Choi. Biodegradable polymer cell carriers with well-defined interconnected spherical pore network. Tissue Eng 7 (1):23-33, 2001. SC-melt molded cell carriers can be made according to R. C. Thomson, M. J. Yaszemski, J. M. Powers, and A. G. Mikos. Hydroxyapatite fiber reinforced poly(alpha-hydroxy ester) foams for bone regeneration. Biomaterials 19 (21):1935-43, 1998. Extrusion—particulate leaching based cell carriers can be made according to M. S. Widmer, P. K. Gupta, L. Lu, R. K. Meszlenyi, G. R. Evans, K. Brandt, T. Savel, A. Gurlek, C. W. Patrick, Jr., and A. G. Mikos. Manufacture of porous biodegradable polymer conduits by an extrusion process for guided tissue regeneration. Biomaterials 19 (21): 1945-1955, 1998.

Freeze-drying (Phase separation) based cell carriers can be made according to H. Lo, Ponticiello M. S., and K. W. Leong. Fabrication of controlled release biodegradable foams by phase separation. Tissue Eng 1 (1):15-28, 1995 and C. Schugens, V. Maquet, C. Grandfils, R. Jerome, and P. Teyssie. Polylactide macroporous biodegradable implants for cell transplantation. II. Preparation of polylactide foams by liquid-liquid phase separation. J Biomed Mater. Res 30 (4):449-461, 1996. Scaffolds manufactured using freeze drying (Phase separation) combined with particulate-leaching can be made according to J. H. de Groot, A. J. Nijenhuis, Bruin P., A. J. Pennings, R. P. H. Veth, J. Klompmaker, and H. W. B. Jansen. Use of porous biodegradable polymer implants in meniscus reconstruction. 1. Preparation of porous biodegradable polyurethanes for the reconstruction of meniscus lesions. Colloid Polym. Sci. 268:1073-1081, 1990 and Q. Hou, D. W. Grijpma, and J. Feijen. Preparation of interconnected highly porous polymeric structures by a replication and freeze-drying process. J Biomed Mater. Res 67B (2):732-740, 2003. Freeze-extraction based cell carriers can be made according to M. H. Ho, P. Y. Kuo, H. J. Hsieh, T. Y. Hsien, L. T. Hou, J. Y. Lai, and D. M. Wang. Preparation of porous cell carriers by using freeze-extraction and freeze-gelation methods. Biomaterials 25 (1):129-138, 2004. Emulsion—Freeze drying based cell carriers can be made using the methods disclosed in K. Whang, C. H. Thomas, and K. E. Healy. A novel method to fabricate bioabsorbable cell carriers. Polymer 36 (4):837-842, 1995.

Scaffolds manufactured using gel casting or solution casting methods can be manufactured using J. P. Schmitz and J. O. Hollinger. A preliminary study of the osteogenic potential of a biodegradable alloplastic-osteoinductive alloimplant. Clin Orthop. (237):245-255, 1988, A. G. Coombes and J. D. Heckman. Gel casting of resorbable polymers. 1. Processing and applications. Biomaterials 13 (4):217-224, 1992 and the methods disclosed in U.S. Pat. No. 5,716,416 (1998). Methods of manufacturing cell carriers using atomized foams can found in H. Lo, Ponticiello M. S., and K. W. Leong. Fabrication of controlled release biodegradable foams by phase separation. Tissue Eng 1 (1):15-28, 1995. Methods using gas Foaming-particulate leaching can be found in Y. S. Nam, J. J. Yoon, and T. G. Park. A novel fabrication method of macroporous biodegradable polymer cell carriers using gas foaming salt as a porogen additive. J Biomed Mater. Res 53 (1):1-7, 2000.

The 3D printing (Theriform™ process) is described in, for example, R. A. Giordano, B. M. Wu, S. W. Borland, L. G. Cima, E. M. Sachs, and M. J. Cima. Mechanical properties of dense polylactic acid structures fabricated by three dimensional printing. J Biomater. Sci Polym. Ed 8 (1):63-75, 1996 and S. S. Kim, H. Utsunomiya, J. A. Koski, B. M. Wu, M. J. Cima, J. Sohn, K. Mukai, L. G. Griffith, and J. P. Vacanti. Survival and function of hepatocytes on a novel three-dimensional synthetic biodegradable polymer cell carrier with an intrinsic network of channels. Ann Surg 228 (1):8-13, 1998. Fused deposition modeling based methods can be found in D. W. Hutmacher, T. Schantz, I. Zein, K. W. Ng, S. H. Teoh, and K. C. Tan. Mechanical properties and cell cultural response of polycaprolactone cell carriers designed and fabricated via fused deposition modeling. J Biomed Mater. Res 55 (2):203-216, 2001. Methods using electrospun nanofibers can be found in W. J. Li, C. T. Laurencin, E. J. Caterson, R. S. Tuan, and F. K. Ko. Electrospun nanofibrous structure: a novel cell carrier for tissue engineering. J Biomed Mater. Res 60 (4):613-621, 2002. Methods using nanofibers with porogen leaching can be found in R. Zhang and P. X. Ma. Synthetic nano-fibrillar extracellular matrices with predesigned macroporous architectures. J Biomed Mater. Res 52 (2):430-438, 2000.

Both natural (e.g., collagen, elastin, poly(amino acids); and polysaccharides such as hyaluronic acid, glycosamino glycan, carboxymethylcellulose; and ceramic based-cell carriers such as porous hydroxyapatite, tricalcium phosphate, and chitosan) and synthetic materials may be used to manufacture the cell carriers of the present invention. In one embodiment, the cell carrier (and/or prosthesis) is constructed of a resorbable material in such as way as to allow room for tissue growth in the cell carrier while eliminating the need for a second surgery to remove the cell carrier (and/or prosthesis). Exemplary synthetic resorbable polymers that may be used to manufacture the cell carriers (and/or prosthesis) of the present invention include, poly(glycolic acid) (PGA), poly(L-lactic acid) (PLLA), poly(D-lactide) (PDLA), poly(D,L-lactide)(PDLLA), trimethylene carbonate, caprolactone, as well as polycaprolactone (PCL), 1,3-propanediol (PDO), polytrimethylene carbonate (PTMC), and/or physical and chemical combinations thereof and their copolymers. In one embodiment, the cell carrier is comprised of a polylactide, which can be a copolymer of about 60-80% L-lactide and about 20-40% D,L-lactide. As examples, one or more of the following polymers can be used: 70/30 L:DL, 85/15 DL:G, 85/15 L:G, 50/50 DL:G, 70/30 L:CL, 70/30 L:TMC, and combinations thereof. At least a portion of these polymers may offer distinct advantages in that their sterilizability and relative biocompatibility have been well documented. Also, their resorption rates can be tailored to match that of desired treatment or new tissue formation. In an illustrative embodiment, the cell carrier can be constructed of 70:30 poly(L-lactide-co-D,L-lactide), which is referenced above as 70/30 L:DL.

In another embodiment, the cell carrier can be constructed of 85:15 poly(D,L-lactide-co-glycolide), referenced above as 85/15 DL:G. Exemplary materials and methods which may be related to making and using all aspects of the present invention are disclosed in, for example, U.S. Pat. Nos. 5,919,234, 6,280,473, 6,269,716, 6,343,531, 6,477,923, 6,391,059, 6,531,146 and 6,673,362, the contents of which are incorporated herein by this reference.

In certain embodiments, any of the scaffolds or cell carrier portions described above may be coated or otherwise formed with apatite using a simulated body fluid (SBF) solution. The SBF solutions may be prepared with ion concentrations approximately 0-10 times that of human blood plasma and can be sterile filtered through a 0.22 μm PES membrane or a similar membrane filter. Methods of making art-recognized SBF solutions and variations thereof for use in the present invention can be found in, e.g., Chou et al. (2004) The Effect of Biomimetic Apatite Structure on Osteoblast Vitality, Proliferation and Gene Expression Biomaterials (In press); Oyane et al. (2003) Preparation and Assessment of Revised Simulated Body Fluids J. Biomed mater Res 65A: 188-195; Murphy et al. (1999) Growth of Continuous Bonelike Mineral Within Porous Poly(lactic-co-glycolide) Scaffolds In Vitro J. Biomed. Mater. Res. 50: 50-58. The scaffolds or cell carriers may be treated with, for example, glow discharge, or argon-plasma etching prior to being soaked in the SBF solution to improve one or more of wettability and affinity for the SBF ions. Different apatite microenvirorments may be created on the scaffold or cell carrier surfaces by controlling one or more of the SBF concentration, components, pH and the duration of the scaffold or cell carrier in each SBF solution. Vacuum or fluid flow (directed or non-directed) can be used, for example, to force the SBF into the pores of the scaffold or cell carrier portion.

It is understood in the art that desired resorption rates of the cell carrier portion will vary based on the particular therapeutic application. It is believed that a cell carrier portion of the present invention having a thickness of 0.1 millimeters to about 5 millimeters should maintain its structural integrity for a period in excess of about two weeks to multiple months, preferably three to six months, before substantially degrading, so that the desired tissue regeneration or treatment can be achieved or optimized.

The rates of resorption of the cell carrier may also be selectively controlled. For example, the cell carrier portion may be manufactured to degrade at different rates depending, for example, upon the rate of recovery of the patient from a surgical procedure. Thus, a patient who recovers more quickly from a surgical procedure relative to an average patient may be administered an agent that for example is selective for the polymeric material of the cell carrier portion and causes the cell carrier portion to degrade more quickly. Or, if the polymeric material is, for example, temperature sensitive or is influenced by electrical charge, the area in which the cell-loaded prosthesis has been implanted may be locally heated or cooled, or otherwise exposed to an agent such as an electrical charge that causes the cell-loaded prosthesis to dissolve at a desired rate for the individual patient.

In addition to resorption rates, certain physical characteristics of the cell carriers may also be considered. Generally, the cell carrier may be constructed to have a relatively large surface area to facilitate, for example, cell attachment. One method of achieving this result may be to create a highly porous polymer foam. In these foams, the pore size can be formed to be large enough so that cells penetrate the pores, and/or the pores may be interconnected to facilitate, for example, nutrient exchange by cells deep within the construct. These characteristics (porosity and pore size) can be or are often dependent on the method of cell carrier fabrication (Mikos, A. G., Bao, Y., Cima, L. G., Ingber, D. E., Vacanti, J. P. and Langer, R. (1993a). Preparation of Poly(glycolic acid) bonded fiber structures for cell attachment and transplantation. Journal of Biomedical Materials Research 27:183-189; Mikos, A. G., Sarakinos, G., Leite, S. M., Vacanti, J. P. and Langer, R. (1993b) Mikos, A. G., Thorsen, A. J., Czerwonka, L. A., Bao, Y., Langer, R., Winslow, D. N. and Vacanti, J. P. (1994). Preparation and characterization of poly(L-lactic acid) foams. Polymer 35:1068-1077; Nam, Y. S. and Park, T. G. (1999a). Biodegradable polymeric microcellular foams by modified thermally induced phase separation method. Biomaterials 20:1783-1790; Nam, Y. S., Yoon, J. J. and Park, T. G. (2000). A novel fabrication method of macroporous biodegradable polymer scaffolds using gas foaming salt as a porogen additive. Journal of Biomedical Materials Research (Applied Biomaterials) 53:1-7).

Pore sizes may in some embodiments generally range from 40 to 400 microns and/or it may be desired in some embodiments to achieve relatively high porosity. Several methods have been developed to create highly porous cell carriers, including fiber bonding (Mikos et al. 1993 above), solvent casting/particulate leaching (Mikos et al. 1993 above; Mikos et al. 1994 above), gas foaming (Nam et al. 2000 above) and phase separation (Lo, H., Ponticiello, M. S. and Leong, K. W. (1995). Fabrication of controlled release biodegradable foams by phase separation. Tissue Engineering 1:15-28; Whang, K., Thomas, C. H., Healy, K. E. and Nuber, G. (1995). A novel method to fabricate bioabsorbable scaffolds. Polymer 36:837-842; Lo, H., Kadiyala, S., Guggino, S. E. and Leong, K. W. (1996). Poly(L-lactic acid) foams with cell seeding and controlled-release capacity. Journal of Biomedical Materials Research 30:475-484; Schugens, C., Maquet, V., Grandfils, C., Jerome, R. and Teyssie, P. (1996). Polylactide macroporous biodegradable implants for cell transplantation II. Preparation of polylactide foams for liquid-liquid phase separation. Journal of Biomedical Materials Research 30:449-461; Nam and Park 1999 above). Of these methods, fiber bonding, solvent casting/particulate leaching, gas foaming/particulate leaching and liquid-liquid phase separation may be used in certain implementations to produce large, interconnected pores to facilitate, for example, one or more of cell seeding and migration. The fiber bonding, solvent casting/particulate leaching and gas foaming/particulate leaching methods may in certain instances exhibit clinically acceptable biocompatibility.

All of the methods described herein, as well as all art-recognized methods for manufacturing cell carriers, may be appropriately modified to remove organic solvents, which may reduce the ability of cells to for example form new tissues in vivo. Some methods of manufacturing the cell carriers of the present invention are described herein by way of example and not limitation.

For example, as set forth above, one technique used for constructing three-dimensional cell carriers is known as “melt molding,” wherein a mixture of fine PLGA powder and gelatin microspheres is loaded in a Teflon® mold and heated above the glass-transition temperature of the polymer. The PLGA-gelatin composite is removed from the mold and gelatin microspheres are leached out by selective dissolution in distilled de-ionized water. Other cell carrier manufacturing techniques may include polymer/ceramic fiber composite foam processing, phase separation, and high-pressure processing.

Another technique for manufacturing cell carriers is known as “solvent-casting and particulate-leaching.” In this technique, sieved salt particles, such as sodium chloride crystals, can be disbursed in a PLLA/chloroform solution which is then used to cast a membrane. After evaporating the solvent, the PLLA/salt composite membranes may be heated above the PLLA melting temperature and then quenched or annealed by cooling at controlled rates to yield amorphous or semi-crystalline forms with regulated crystallinity. The salt particles can be eventually leached out by selective dissolution to produce a porous polymer matrix.

Yet another technique for manufacturing cell carriers of the present invention is known as “fiber bonding,” and can involve preparing a mold in the shape of the desired cell carrier and placing fibers, such as polyglycolic acid (PGA) into the mold and embedding the PGA fibers in a poly(L-lactic acid) (PLLA)/methylene chloride solution. The solvent can be evaporated, and the PLLA-PGA composite can be heated above the melting temperatures of both polymers. The PLLA can then be removed by selective dissolution after cooling, leaving the PGA fibers physically joined at their cross-points without any surface or bulk of modifications and maintaining their initial diameter. Fiber bonding may in some embodiments be particularly useful for fabrication of thin cell carriers.

Another technique may be solid freeform fabrication (SFF), which refers to computer-aided-design and computer-aided-manufacturing (CAD/CAM) methodologies that have been used in industrial applications to quickly and automatically fabricate arbitrarily complex shapes. SFF processes construct shapes by incremental material buildup and fusion of cross-sectional layers.

In these approaches, a three-dimensional (3D) CAD model is first decomposed, or “sliced,” via an automatic process planner into thin cross-sectional layer representations that are typically 0.004 to 0.020 of an inch thick. To build the physical shape, each layer is then selectively added or deposited and fused to the previous layer in an automated fabrication machine. These and other cell carrier manufacturing techniques are discussed in U.S. Pat. No. 6,143,293, the entire contents of which are incorporated herein by this reference.

In accordance with another aspect of the present invention, the cell carrier portion may comprise a substance for cellular control, such as one or more of a chemotactic substance for influencing cell-migration, an inhibitory substance for influencing cell-migration, a mitogenic growth factor for influencing cell proliferation, a growth factor for influencing cell differentiation, factors which promote angiogenesis (formation of new blood vessels), and combinations thereof. Cellular control substances may be located at one or more predetermined locations on the cell carrier. For example, substances that generally inhibit or otherwise reduce cellular growth, treatment, and/or differentiation may be located on one or more surfaces of the cell carrier (e.g., surface that will not be in proximity to the area of intended treatment). Similarly, substances that generally promote or otherwise enhance cellular growth, treatment, and/or differentiation may be located on one or more surfaces of the cell carrier (e.g., the surface that will be in proximity to the area of intended treatment). Additionally, the inhibiting and promoting substances may be interspersed through the cell carrier at predetermined locations to help influence rates of cellular growth at different regions over the surface of the cell-loaded prosthesis.

One implementation of an appropriate cell carrier portion of the cell-loaded prosthesis may be pre-formed into specific shapes, configurations or sizes that conform to the shape of the cell-loaded prosthesis by the manufacturer (e.g., by MacroPore Biosurgery, Inc.) before packaging and sterilization. However, in other implementations, a pre-formed cell carrier portion can also be shaped at the time of surgery by bringing the material to its glass transition temperature, using heating iron, hot air, heated sponge or hot water bath methods. In a certain embodiment, the cell carrier could be cut and mechanically press-fit into a containment portion and held in place by the resulting interference fit. In another embodiment, the containment portion could be heated to glass transition temperature to revert to a predetermined and preformed geometry, resulting in a clamping of the cell carrier, resulting in stabilization. In yet another embodiment, the cell carrier may be affixed to the containment portion by one or more appropriately sized mating resorbable or non-resorbable screws, tacks or pins inserted through one or more of apertures in the containment portion. Resorbable screws, tacks or pins can be obtained from MacroPore Biosurgery, Inc. (San Diego, Calif.). Alternatively, once inserted into an intended area of tissue treatment or regeneration, the influx of water to the area could expand the cell carrier and shrink the containment portion or shrink the cell carrier and expand the containment portion, resulting in fixation. In other embodiments, the cell carrier and/or containment portion may be affixed to each other using polymer solutions, solvents or appropriate glues.

3. Illustrated Embodiments of Cell-Loaded Prostheses that may be Formed with Cell Carriers

Following are exemplary embodiments of cell-loaded prostheses that may be formed (e.g., coated) with cell carriers. The preceding methods, structures, compositions, and discussion may be combined, in any operable fashion, in whole or in part, with any modification as may be apparent or recognizable to one skilled in the art, to the extent compatible and not mutually exclusive, with the following.

A flexible tubular body according to the present invention has been found to be suited for use as a cell-loaded prosthesis for intraluminal or transluminal implantation in blood vessels or other similar organs of the living body. The tubular body can be inserted into place in the organism in a contracted state, i.e., with reduced diameter. After the tubular body has been inserted into position it can be subjected to expansion and can stay in place in an expanded state by self-fixation if the diameter of the body in unloaded condition is selected somewhat larger than the diameter of the surrounding wall. This construction results in a certain permanent pressure of engagement against the inner wall so as to ensure good fixation.

To obtain the desired function the axially directed angle between crossing elements can be greater than about 60 degrees, and can in some embodiments be obtuse, i.e., more than about 90 degrees. This state of the body refers to its state in radially unloaded condition.

The crossing thread elements can be arranged in such a manner as to form a sort of braided configuration which may be varied as desired and for example imitate some known type of weaving, for example according to the principle of a plain weave. This can impart to the tubular body the necessary stability. If the number of elements in the flexible tubular body is designated n then n can vary, e.g., from about 10 and up, for example to about 50. The elements of the tubular body can be arranged symmetrically, i.e., the number of elements in each direction of a winding can be (n/2). It should be observed that in this connection when referring to the number of elements in the tubular body reference can be had to elements intended to maintain the supporting function of the body. The number of elements n can be selected in accordance with the diameter of the body, the diameter of the element, the material of the element or other factors. Generally, the greater the diameter of the body with a given element material, the more elements should be used to give the necessary stability of the body.

The radially contracted cell-loaded prosthesis, which e.g. can be inserted through the wall of the vessel at a distance from the implantation site, will be fixed without the need for conventional removal of the parts of the organ to be replaced. In this manner the blood flow can be maintained even during the implantation which calls for a short period of time. The cell-loaded prosthesis in accordance with certain embodiments need not be stitched to the vessel and already after a few days it can be definitely fixed to the body by means of natural tissue growth.

The flexible tubular body can be brought to expand radially in several ways. It may be preferred for the body to have the property of entering into radially expanded and unloaded position by itself. The expanded state of the body may be dependent on the inherent rigidity of the thread elements, but it may also be controlled by elastic strings, bands or membranes, or expandable means such as a balloon catheter which are arranged in connection to the mantle surface of the body and extend axially along same. By their elasticity these strings, bands or membranes result in axial traction of the body, i.e., to bring same to take an expanded state.

An alternative way of imparting properties to the body through which it tends to take a radially expanded position can be to attach the elements to each other at the points of crossing thereof in a suitable manner, for example by some form of welding, gluing or the like.

The elements forming the flexible tubular body should be made of a medicinally acceptable material, for example plastic or metal or non-metallic synthetic materials, and can possess certain springiness or rigidity combined with suitable elasticity. The elements may be built up as monofilaments, for example polypropylene, dacron or other suitable plastic or constituted by a composite material. They may also be made from some suitable medicinally acceptable metal.

The free ends of the thread elements of the tubular body can be modified or protected in several ways. An alternative in which no free ends at all are present may be the alternative to make the tubular body as a whole of one coherent element. The alternative which may be most closely related to that may be the case where the free ends of a body resulting from severing a long string are connected with U-shaped members which are attached to the ends of the elements pair-wise in a suitable manner, for example heat welding, gluing or the like. In this manner elements of the same direction of winding or elements of the opposite direction of winding can be attached to each other two and two.

An alternative to these embodiments can be to weld together the points of crossing in a ring around the material by electric resistance heating or the like before severing the string, severing then taking place adjacent to and just outside the welding site. The ends then extending outside the welding area may be folded inwardly towards the interior of the body with light plastic deformation, for example through controlled heating. Yet another alternative consists in bending the free ends of the elements to form loops.

In accordance with one embodiment of the present invention, the tubular body can be suited for use as a so-called graft. In this case the body may function as a graft namely if it is made of elements of such character as to impart by themselves the desired density and porosity to the body to function as a graft whereby at least a number of the elements may be made of polyfilament materials or the like. The alternative of the elements themselves imparting the desired density to the body can be to apply some sort of surface layer to the body, for example of plastic or other suitable material. By applying such surface layer the crossing points may at the same time be fixed as indicated above so as to make the body tend to take an expanded position.

Outside or inside or amalgamated with the body there may also be arranged a separate sleeve or membrane. This can be constituted by a stocking of porous web surrounding the body which is implanted together with the body. In this case the stocking may, either by stretchability in the web or by overlapping folding or in another manner, for example by being built up in accordance with the same principle as the body from a plurality of thread elements, be adjustable to the body in connection with the expansion thereof. It may also be possible to conceive the use of some form of tricot type product or crimped fiber textile. When using such a separate member it may be preferred that it is axially fixed relative to the body so as to end up in the right position when applied in a large vessel or the like.

The expansion or contraction of the tubular body can be provided by a device with means which are arranged to elongate or shorten the body. Such means may be designed in many ways, for example so that their construction allows axial movement of the ends of the body relative to each other to reduce or increase the diameter of the body. The device should include gripping members capable of gripping the ends of the body and axially moving the same relative to each other. The gripping members should be arranged so as to be releasable after the application of the body at the desired site so that the device except for the body can be removed from the site. Alternatively, the device may include a flexible tube within which the tubular body can be placed in contracted state, and operating members by means of which the body under expansion thereof can be pushed out of the tube to be applied at the desired site.

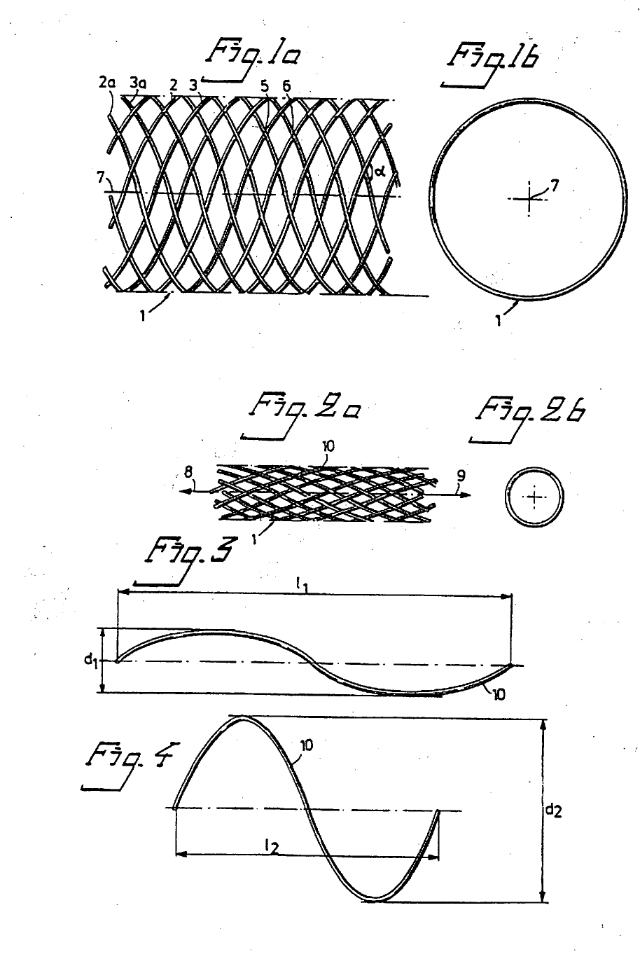

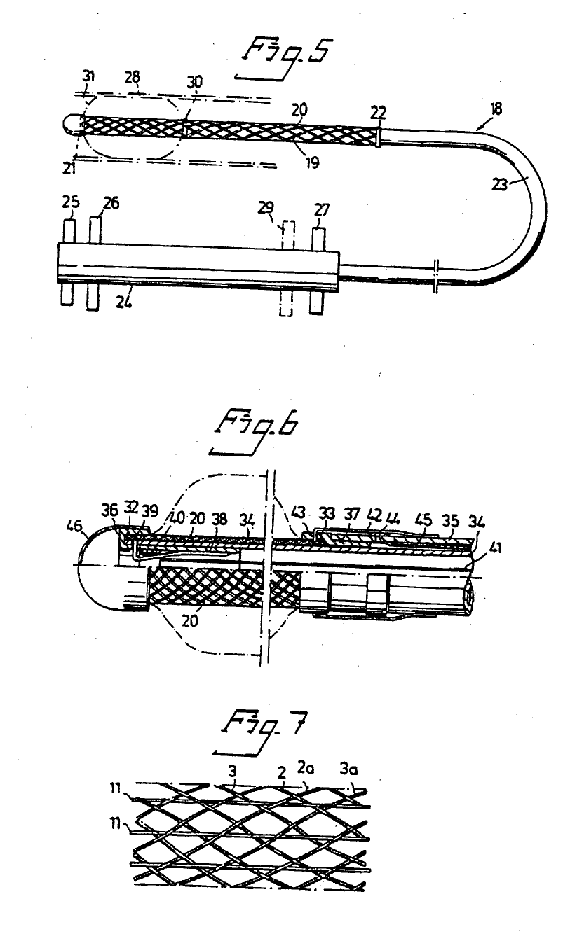

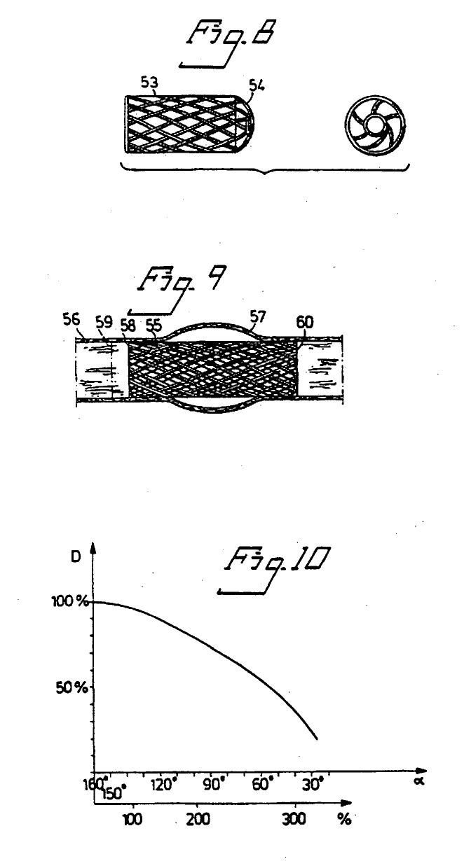

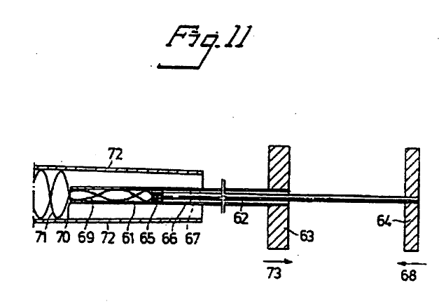

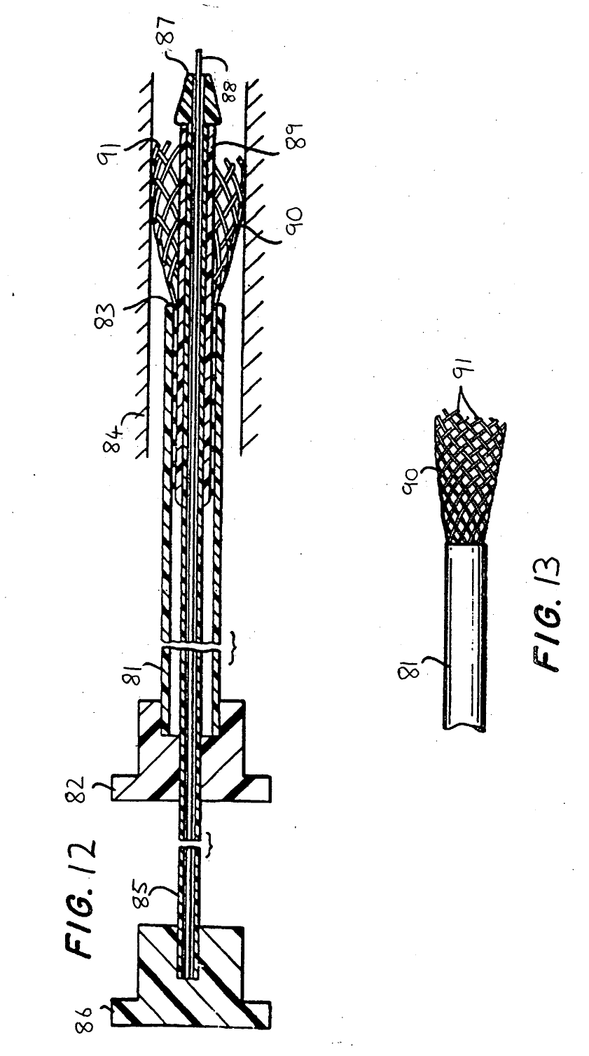

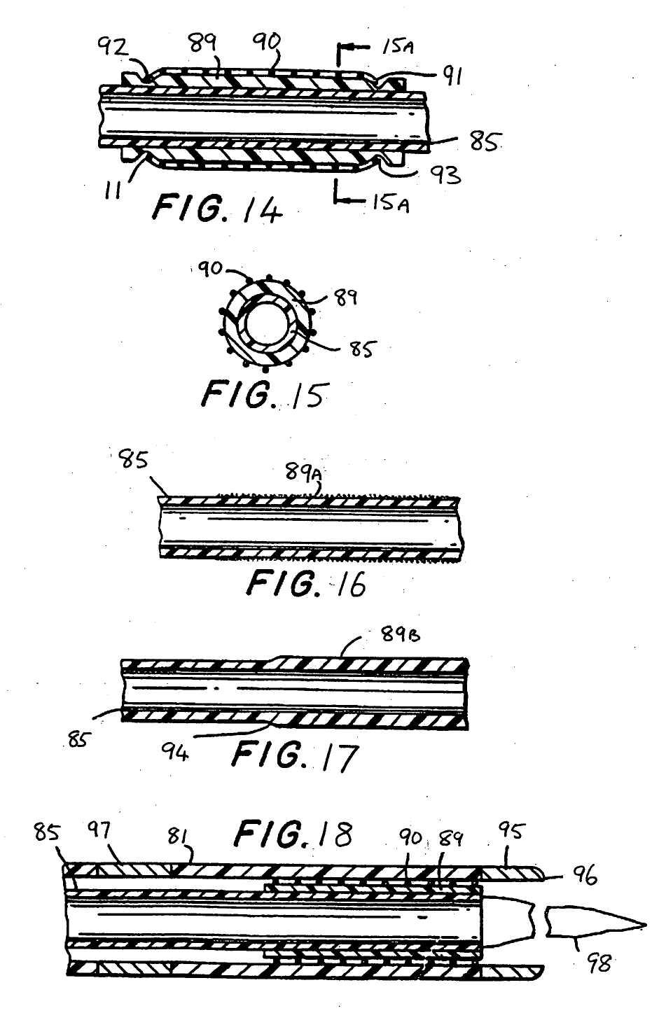

In FIGS. 1A and 1B there is shown an exemplary inventive cell-loaded prosthesis in the form of a cylindrical tubular body generally designated 1. As is clear from FIG. 1A the mantle surface of body 1 can be formed by a number of individual thread elements 2, 3 etc. and 2a, 3a etc. Of these elements 2, 3 etc. extend in helix configuration axially displaced in relation to each other having the center line 7 of body 1 as a common axis. The other elements 2a, 3a extend in helix configuration in the opposite direction, the elements extending in the two directions crossing each other in the manner indicated in FIG. 1A.

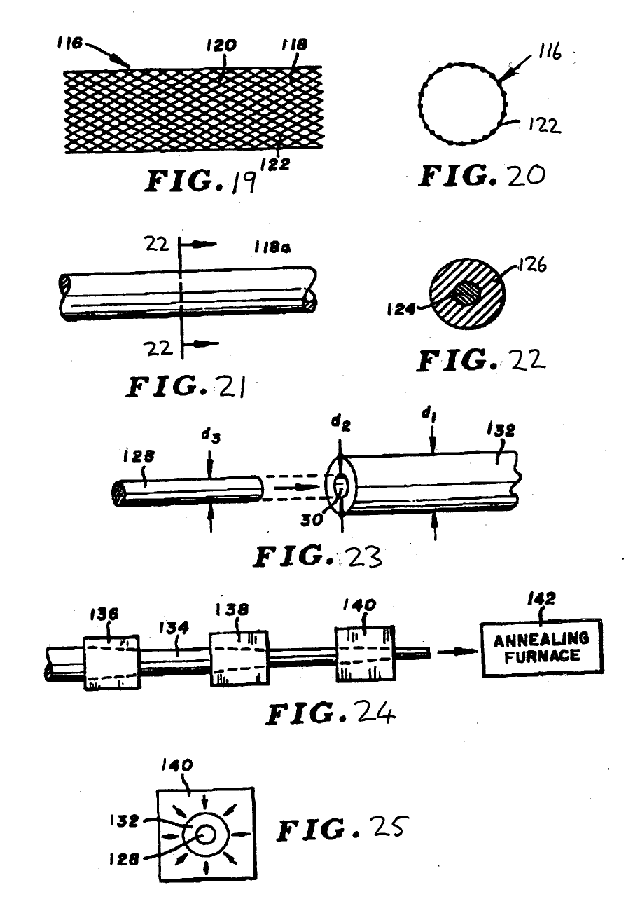

The diameter of a tubular body built up in this manner can be varied if the ends of the body are axially displaced relative to each other in the direction of the center line 7. In FIG. 2A there is illustrated how the tubular body 1 according to FIG. 1A has been given reduced diameter by moving the ends 8, 9 away from each other in the direction of the arrows. FIG. 1B shows the diameter of the tubular body in an expanded state, whereas FIG. 2B shows the diameter of body 1 in contracted state after the ends 8, 9 thereof have been moved away from each other.

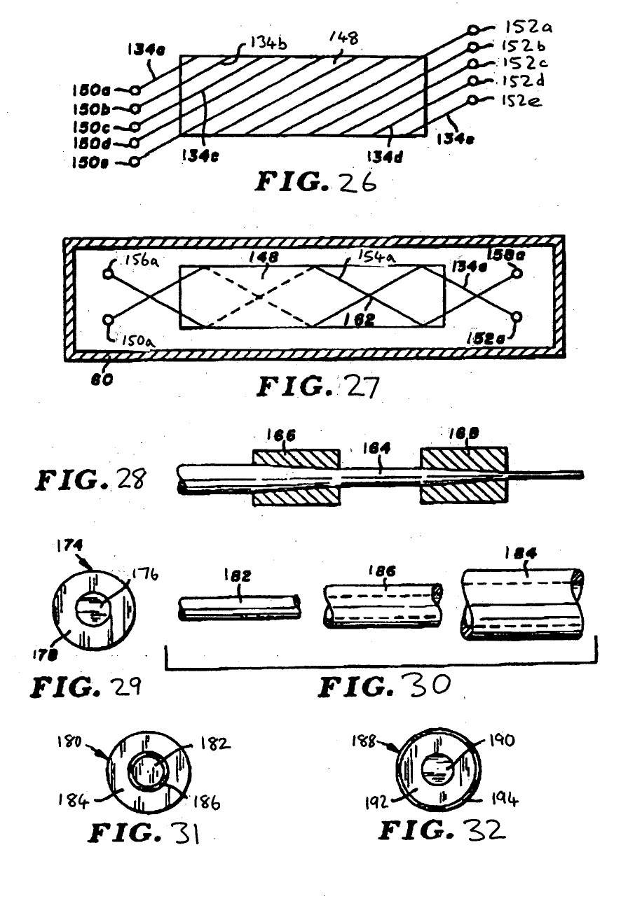

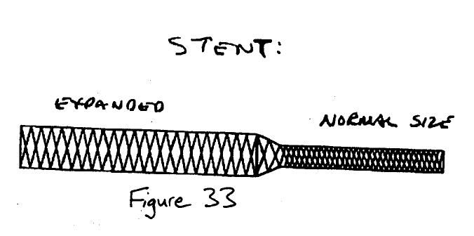

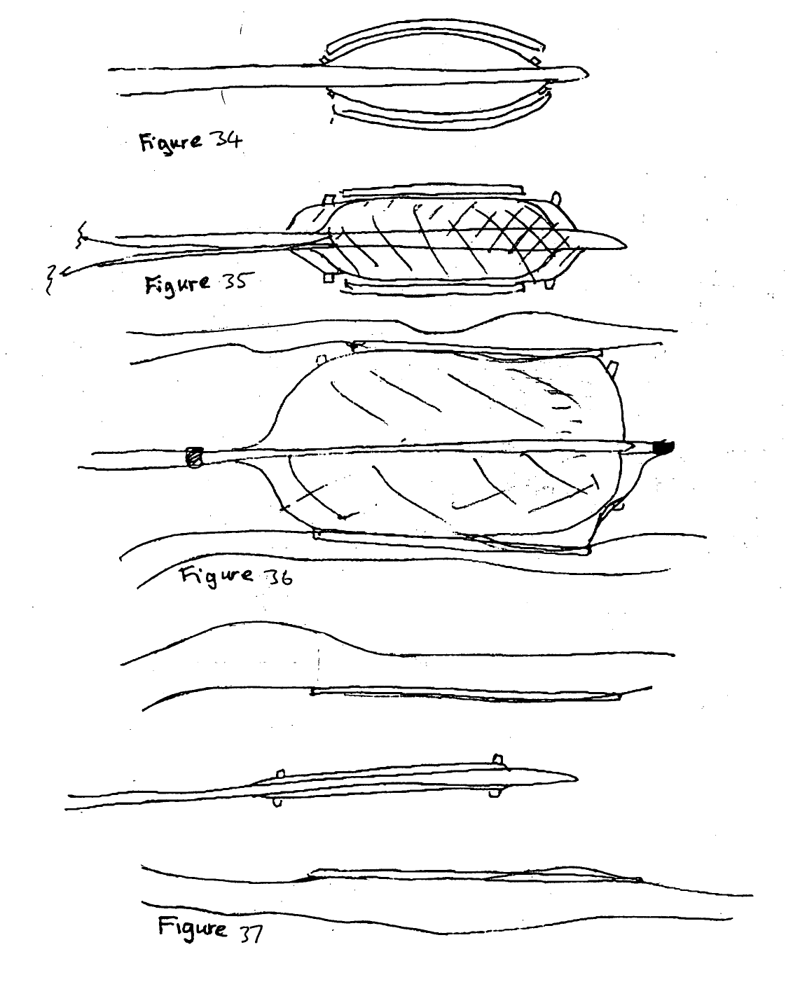

FIGS. 3 and 4 show a detail picked from FIGS. 1 and 2, more particularly one single thread element of the tubular body 1 and how its helix configuration will be changed in connection with the change of the length of the tubular body 1.

In FIG. 3 the individual element 10 corresponding to element 10 of FIG. 2A is shown. The diameter of the helix is d₁ and the length of the element is l₁. In FIG. 4 the same element 10 is shown after the tubular body has been expanded to the state shown in FIG. 1A. The diameter of the helix has now increased and is designated d₂, whereas the length has decreased and is designated l₂.

The tubular body 1 can be expanded in a number of ways. As previously mentioned it may be preferred that the body inherently has the property of taking expanded position by itself in unloaded condition. In the present disclosure the expression “expanded position” refers to radial expansion, i.e., a state with a large diameter of body 1. The self-expanding property can be obtained, for example, by providing the body with strings or bands extending parallel and axially with the mantle surface of the body. An example of such embodiment is shown in FIG. 7 where the tubular body 1 can be provided with axial strings or bands 11. In such an embodiment wherein strings or bands 11 are used, the strings or bands 11 can be suitably made of an elastic material and can be fixed to the elements of the tubular body 1 in a suitable manner and with the body in expanded state. Now, if the tubular body 1 can be axially elongated by moving the two ends thereof from each other the elastic strings or bands 11 will be stretched. After removal of the tensile force from the body 1 the elastic strings or bands 11 will compress the body 1 in an axial direction resulting in a corresponding increase of the diameter of the body. The strings or bands 11 may in a resorbable embodiment comprise a resorbable material so that by changes in temperature thereof they may be brought in and out of a glass transition phase to thereby increase or decrease their lengths. For example, they may be formed so that when they are heated their lengths shorten.

The tubular body 1 can be provided with the same tendency to take expanded position by fixing the elements 2, 3 etc.; 2a, 3a etc. at the crossing points 5, 6 (FIG. 1), as previously mentioned. Another way of providing this effect can be to provide for an interior or exterior tubular elastic member, for example of a thin elastomer, which can be attached to at least both ends of the tubular body.

In FIG. 5 there is shown a device generally designated 18 to enable insertion of an expandable body, such as the tubular body 20, in contracted and elongated state at the desired site of for example a blood vessel. To the extent compatible, the device may be used with any implant/stent set forth herein. The tubular body 20 surrounds the forward tubular part 19 of apparatus 18 and can be attached at both ends thereof to gripping means 21 and 22. The forward tubular part 19 of the apparatus can be connected to an operational member 24 through a flexible tubular means 23. By means of operational elements 25, 26 and 27 of the operational member 24 the gripping means 21 and 22 can be controlled in a desired manner.

In FIG. 5 there is shown diagrammatically how apparatus 18 with the contracted tubular body 20 has been inserted into for example a blood vessel which in the figure is shown with dashed lines and designated 28. Operational member 24 can be connected with gripping member 22 in such a manner that when the operational means 26 can be moved forwardly to position 29 shown with dot and dash lines a gripping member 22 can be displaced in a corresponding manner to the dot and dash line position 30. As a result the end of tubular body 20 has been moved from position 22 to position 30, whereas in this case the other end of the body remains in position 21. At the same time the diameter of body 20 has increased and when the end has reached position 30 the body 20 can be expanded, i.e., it has been brought into contact with the interior wall of the vessel and has taken dash-dotted line position 31. Since both ends of the tubular body 20 are still held by members 21, 22, body 29 in expanded state takes a balloon-like shape.

Operational means 27 can also be connected with the gripping member 22 by means of a part, for example a wire, running in tubular member 23. In this manner gripping member 22 in its position 30 can be maneuvered by axial displacement of operational member 27 to release the end of the body 20. In the same manner maneuvering means 25 which can be connected to gripping member 21 can release the forward end of the tubular body from gripping member 21 by axial displacement thereof. The ends of the elastic body 20 are thereby immediately subjected to movements relative to each other to provide for expansion and the cell-loaded prosthesis takes its expanded cylindrical shape in the interior of the blood vessel.

In FIG. 6 there is shown more in detail and in an enlargement the construction of the forward tubular part 19 of device 18. The tubular body 20 with both its ends 32 and 33 surround a thin-walled flexible tube 34 running inside and concentrically to an outer flexible tube 35, the two tubes of which form the tubular member 23 in FIG. 5. At the front part of the inner tube 34 an annular member 36 can be arranged, into which the end 32 of tube 20 can be inserted. In a corresponding manner the end 33 of tube 20 can be inserted into an annular member 37 which can be axially displaceable in relation to the tube 34 surrounded by ring 37. At the front part of tube 34 there can be provided an interior gripping member or latch 38. Latch 38 which can comprise spring steel, has a forward pointed part 39 bent under about right angle. This part 39 extends radially outwardly through a hole in tube wall 34. It can move in radial direction under the influence of a ring 40 which can be axially movable and arranged inside tube 34. Ring 30 can be connected to a wire 41 through which by axial displacement latch 38 can be moved in a radial direction. In FIG. 6 latch 38 is shown in such position that its pointed part 39 has perforated the end 32 of body 20 and thus maintains the end in position.

In the corresponding manner another latch 42 can be arranged to hold from outside the end 33 of the tubular body 20 by its pointed part 43. This latch 42 which can be attached to the outside of tube 35 can be moved in radial direction by means of a ring 44 arranged about tube 35 and attached to a cable 45 extending between tubes 34 and 35. Cables 44 and 45 are connected to the operational means 25 and 27, respectively, in FIG. 5.

When the attached and axially extended tubular body 20 shall be released from the remaining part of the device after the axial expansion of the body, this takes place by releasing the pointed parts 39, 43 of latches 38 and 42, respectively, from the ends of the tubular body 20 by actuating rings 40 and 44 through operational members 25 and 27 via cables 41 and 45, so as to deflect latches 38 and 42. The ends 32 and 33 of the body 20 will then be released by axial displacement of the tubular part 19 of the apparatus. As is clear from FIG. 6 the front end of the apparatus can be protected by a hub or casing 46 attached to ring 36.

As previously indicated the expansible tubular body finds several applications within surgery. For example, in the embodiment shown in FIG. 1 it can be utilized for supporting vascular walls. In FIG. 8 there is shown a modified embodiment of the flexible tubular body. In this embodiment the body consists of a cylindrical circular part 53 which at one end thereof changes to a diminishing part or end 54 also built up from thread elements. This device has been found to be suitable for use as a sieve or filter to prevent thrombosis. The device shown in FIG. 8 can be applied at the desired location within a blood vessel, for example Vena Cava Inferior, for the purpose of preventing lung emboli.

In FIG. 9 there is shown a tubular body for use as a graft. In this case body 55 may have a denser or a much denser wall than the embodiment shown in FIGS. 1 and 2. This denser wall can be obtained by weaving an elastic yarn between the supporting thread elements 2, 3 etc.; 2a, 3a etc. of FIG. 1. In this manner a wall having a controlled porosity can be obtained. This tubular body having a more or less porous wall is thus a sort of expansible graft that may have a versatile use.

In the application shown in FIG. 9 the body 55 can be implanted into for example an aorta 56 wherein there is an aneurism 57 in the form of a widening of the vascular wall. In view of the fact that the expansible body or graft 55 can be inserted at a distance from the damaged location of an aorta and then located in the middle of the aneurism the latter will be bridged and need not be operatively removed. In FIG. 9 it is also indicated that the aorta can be a conical blood vessel. Therefore, the procedure in this case will be that the cell-loaded prosthesis in the form of a graft can be inserted with an instrument, for example in accordance with FIG. 5. After being located, the graft or body 55 can be expanded. In view of the conical configuration of the aorta the surgical techniques will be as follows.

The front end 31 of graft 55 according to FIG. 5 can be inserted somewhat further into the aorta than the location it shall take after terminated operation. This position 59 is indicated in FIG. 9 with the dotted line. The other end 22 of the axially extended graft 55 according to FIG. 5 can be carried up to the final position corresponding to position 60 of FIG. 9 before the radial expansion. Since this part of the aorta has a somewhat smaller diameter than the diameter in front of the aneurysm as seen upstream in relation thereto the cell-loaded prosthesis cannot expand more than the dimension corresponding to the diameter at end 60. This is, however, alleviated by then moving the other end of the graft 55 by means of the front part of the instrument from position 59 to position 58 so that this end of the graft can expand sufficiently to engage this part of the vascular wall.

In FIG. 11 there is shown another embodiment of the assembly for use in expanding the tubular body. This assembly constitutes a flexible instrument intended to introduce the tubular body in contracted state into for example a blood vessel and then to expand the body when located therein. The parts of the instrument consist of an outer flexible tube 61 and a concentric also flexible inner tube 62. At one end of the outer tube an operational member 63 can be arranged. Another operational member 64 can be attached to the free end of inner tube 62. In this manner the inner tube 62 can be axially displaceable in relation to the outer tube 61. At the other end of inner tube 62 a piston 65 can be attached which when moving runs along the inner wall of outer tube 61.

When the instrument is to be used the tubular expansible body 69 in contracted state can first be placed inside tube 61, the inner tube 62 with the piston 65 being located in the rear part 66 of outer tube 61. Dashed lines show the starting position of piston 65 at 67 in FIG. 11. In this manner part of tube 61 can be filled with the contracted tubular body 69 in the starting position.

During implantation the flexible tubular part of the device can be inserted into the location of a blood vessel intended for implantation. Member 64 is then moved in the direction of arrow 68, the contracted body 69 being pushed out through end 70 of tube 61, the part of the tubular body 69 leaving the tube end 70 expanding until in its expanded position 71, it is brought to engagement with the interior of vascular wall 72. The tubular body 69, 71 is for the sake of simplicity shown in FIG. 11 as two sinus-shaped lines. To the extent that the expanded body 21 comes into engagement with vascular wall 72 moving member 63 in the direction of arrow 73 moves the tube end 70. The piston 65 pushing against one end of the body moves the contracted body 69. Thus, the implantation takes place by simultaneous oppositely directed movements of members 64 and 63, the displacement of member 64 being larger than that of member 63. When the contracted body 69 has been fully removed from the tube 61 the expansion is terminated and the instrument can be removed from the location of the operation.

The embodiment according to FIG. 11 can be suitable for implantation of helices with very small diameters. As an example a tubular expansible body may comprise crossing thread elements, the contracted diameter of the body being only 2 mms and the expanded diameter 6 mms. It is also fully conceivable to implant expanded bodies with even smaller diameters. The instrument according to FIG. 11 may also advantageously be used for implantation of bodies in the form of grafts of a very large diameter.

In implantation of long bodies it is conceivable that the resistance in displacing the same in tube 61 becomes too high. In this case it may be suitable to replace piston 65 at the front end of tube 62 with movable jaws or latches which operate in such a manner that when tube 62 is brought forward in the direction of arrow 68 the latches engage the inner side of body 69, the body being brought forward. When tube 62 is brought back in the direction of arrow 73 the latches are released. In this manner a pump-like motion of tube 62 can move body 69 forwardly.

Many embodiments of the different members shown in FIG. 11 are, of course, conceivable. Thus, it may be possible for example to simplify implantation for the surgeon by controlling the relative motion between members 63 and 64 in a mechanical manner.

The expansible body should possess certain elastic properties in order to enable successful implantation. For example, when the body is inserted to keep blood vessels open or is implanted as a blood vessel cell-loaded prosthesis it should in accordance with one aspect of the present invention have elastic properties which are as similar as possible to those of the blood vessel of the living body. The body should also remain fixed against the surrounding organ, for example the blood vessel, during the stress and strain to which the organ may be subjected. The body should at the same time be elastically resilient radially and axially so as to have for example sufficient adaptability to follow pulsation of the blood or the bending of a limb. The body should also have sufficient inherent rigidity so as to maintain its shape at for example external pressure and must have sufficient strength to resist internal pressures.

In order to obtain these properties it may be suitable to carefully select and adapt materials and dimensions on the thread elements of the body to the actual area of application. In addition to the obvious requirement that the material of the thread elements shall be compatible with the tissue, i.e., inter alia result in minimum reaction of rejection, non-toxic and enable cell growth, it may be generally the that the material should be rigid and elastic and not plastically deformable to any significant extent. The material may for example be monofilaments of polyesters, polyurethanes, polycarbonates, polysulphides, polypropylene, polyethylene, polysulphonates, stainless steel, and silver. The diameter of the monofilament should suitably lie in accordance with one aspect of the invention within the range of about 0.01 to about 0.5 mms, although other diameters may be used in modified embodiments.

In certain cases it may be important that the angle alpha between the thread elements of the body, for example between 2 and 2a of FIG. 1A, when the body is expanded or is in an unloaded or nearly unloaded state is sufficiently large, inter alia, to meet the above requirements.

It has been found that the greater the angle alpha the higher the stability of the body under external pressure. The ideal from this point of view would be 180 degrees, which may not be practically possible. The angle as shown in FIG. 1A can be about 160 degrees, which may in some instances be close to the upper limit.

In order to change the diameter of the body it may be required, as indicated, that both ends of the body are axially displaced relative to each other. In FIG. 10 there is shown the general relation between this movement. The change in percent in diameter when the ends are moved away from each other has been plotted along the y-axis and along the x-axis the corresponding change in percent in length expressed as elongation. Along the x-axis there has also been plotted the angle alpha as a function of the diameter of the body.

As is shown from FIG. 10 the relative diameter reduction can be small at the outset of the elongation process and the diameter has been reduced to the order of 90% when the elongation is 100%, referring to the starting position where the angle alpha is as close to 180 degrees as is practically possible. At an elongation of 200% the diameter reduction can be 75% corresponding to an angle alpha of 100 degrees. The diameter reduction will then be accelerated at increasing elongation. Thus, an elongation increase from 250 to 300% results in a diameter reduction from 60% to 30%, i.e., a relatively large diameter change at a relatively small elongation. Within this range the angle can be reduced from about 70 degrees to about 40 degrees. As indicated above it may in some cases be desirable that the expanded body takes a position which may be as far to the left on the curve of FIG. 10 as possible, i.e., the angle alpha should be as large as possible. Since the implanted body must engage against the vascular wall with certain pressure in order to remain fixed the diameter of implantation should be smaller than the diameter at free expansion.

When using expansible bodies according to the invention for implantation in blood vessels or other tubular organs the necessary expansion forces may be provided for example by elastic means, such as longitudinally extending elastic strings fixed at the crossing thread elements of helix configuration. By selecting a large angle alpha when the elastic means are fixed to the elements the requirements previously mentioned may be met in a simple manner.

The reason why a large value of the angle alpha is often desirable is the fact that the elastic properties of the cell-loaded prosthesis may be impaired with decreasing angle. Under for example exterior pressure in a radial direction the resistance to deformation can be small and there can be a risk for local axial displacement between the cell-loaded prosthesis and vascular wall, which can prevent cell growth at the site of displacement. Another reason for selecting a high value of the angle alpha can be in those cases where a high expansion ratio is desired, i.e., a high ratio between diameter of the expanded body and the diameter thereof in contracted state. In order to obtain for example expansion ratio over 2 up to about 3 the angle alpha should exceed about 120 degrees. The selection of the angle alpha also varies depending on the material of the thread elements of the cell-loaded prosthesis. If a plastic material has been selected too small an angle alpha results in too high resiliency in the radial direction. In some other cases it may, however, be desirable to select a smaller angle alpha, namely in those cases where pronounced radial yield is desired.

Another case where a high value of the angle alpha might be desirable is applications wherein the cell-loaded prosthesis as applied will be subjected to a bending. One application of the implants of the present invention is within vessels in connection with neurological applications. The resistance to flattening of the cell-loaded prosthesis will thus be higher the larger the angle alpha. Thus, it may be suitable to select an angle alpha which is more than about 60 degrees, and an obtuse angle alpha could be particularly suitable. To provide for high resistance to external pressure or to enable high expansion ratios it may be preferred to select an angle alpha of at least about 120 degrees.

From FIG. 10 it is clear that the body must be highly extended when using large angles alpha. To enable intraluminal or transluminal implantation through passages of small diameters the elongation starting from large angles alpha may be substantial and can be up to 300% and even more.

When implanting for example vessel prostheses or similar devices, to keep blood vessels open, it may be as a rule desirable to reach a pressure against the surrounding vascular wall which may be at least about 100 mm Hg. There can be also a highest pressure which should not be exceeded. This highest pressure varies from case to case but should not exceed for example about 500 to 1000 mm Hg when used as a vascular cell-loaded prosthesis. If the desired pressure will be provided by longitudinally extending elastic members or an elastic sleeve or membrane the necessary pressure for fixation can be obtained with reasonable forces when selecting a large angle alpha which can be advantageous. Thus, calculations show that in smooth cylindrical engagement between vascular cell-loaded prosthesis and surrounding vascular wall there may be required a total force of a few Newtons (.about.0.1-0.2 kp) to obtain fixation if the angle alpha is 150 degrees-170 degrees. This fact also contributes to the reduced risk of displacement of the implanted cell-loaded prosthesis under external pressure since the frictional forces arising are sufficient to prevent such displacement. If the angle alpha is for example 45 degrees there may be, however, required a force of about 10-20 Newtons (1-2 kp) which can be practically disadvantageous.

In order that the cell-loaded prosthesis of the invention shall operate in a satisfactory manner, inter alia to give the necessary fixation when applied, such requirements should be met in regard to the elastic material resulting in the necessary expansive force. The material must also result in acceptable adherence to the thread elements of the body and should, of course, be biologically acceptable for implantation. The material shall thus have a low module of elasticity and should present a linear relation between force and elongation at least up to 250-600% elongation and should not possess significant hysteresis.

There is a group elastomers meeting the above requirements which has been found suitable for use in manufacturing expansible bodies according to the invention. Such elastomers are included within the group of materials called segmented polyurethanes (PUR), several of which are commercially available under trade names such as Pelethane (UpJohn), Biomer (Ethicon), Estane (BF Goodrich). These materials can be dissolved in suitable solvents to form solutions, from which thin elastic bands or thin-walled tubes can be prepared for attachment to the supporting thread elements of helix configuration forming the framework of the body.

When using a cell-loaded prosthesis in accordance with one aspect of the present invention as grafts or vascular prostheses the wall of the cell-loaded prosthesis, as previously mentioned, can be porous, thin and compatible with tissue and be composed so as to enable growth of natural tissue, inter alia neointima. In addition to resorbable plastics, segmented polyurethanes (PUR) are also suited for use to form such walls since the properties can be combined with the requirement of a wall having a very high elasticity. Such walls, regardless of the specific material or materials used, may be prepared in the form of a thin tube consisting of fibres of segmented PUR formed by extrusion from a solution of PUR. The fibres are attached to each other at the crossing points and the wall can be made with the desired porosity by suitable adjustment of for example fiber thickness and density. The resulting tube can surround the body or can be attached to the inside thereof. Alternatively, the thread elements of the body can be amalgamated with the tube material, suitably when preparing the tube.

In order to impart the desired expansional force to a vascular cell-loaded prosthesis bands of PUR, resorbable plastic, and/or other materials may be combined with suitable porous wall material which can consist of monofilaments or multifilaments interwoven between the thread elements of the body or which can consist of a porous elastic wall prepared according to what has been described above.

It may be suitable to make the body or its bonds, sleeve or membrane from a biologically degradable material, for example polylactide.

In another illustration of the present invention the type of stent to be deployed or retracted by an instrument according to the invention can be a self-expanding braided stent, such as described above. However, it is to be understood that the instrument for deployment and/or retraction may be used for the placement of any expansible stent having a configuration and dimensions which enable it to be releasably held between the grip member and outer sleeve of the instrument. In accordance with on aspect of the present invention, the below-discussed stent comprises a biologically degradable (e.g., resorbable) material.

In an embodiment of the invention wherein the core can be hollow, proper positioning of the instrument in a body canal may be facilitated. Thus it may be possible to pass a guide wire into and along the body canal and pass the instrument over the guide wire until it is properly positioned in the body canal. In this embodiment the instrument includes an elongated, flexible, steerable guide wire located within and along the axis of the core. When the instrument is positioned in the body canal the guide wire may be retained within the instrument until the stent is deployed at the desired location and withdrawn together with the instrument or, alternatively the guide wire may be withdrawn prior to deployment of the stent so that correct positioning of the stent, while still within the instrument, may be verified, for example, by endoscopic or fluoroscopic means.

To facilitate movement of the core relative to the outer sleeve, the core can have a handle attached to its proximal end and the proximal end of the outer sleeve can terminate in a flange or handle.

It is to be understood that, as used herein, the term “proximal” means the end or part nearest to the operator of the instrument and the term “distal” means the end or part furthest from the operator. Thus the front end of the instrument which enters the body canal is the distal end. A significant feature of the instrument is the grip member and the significance of this feature can be that it enables both deployment and retraction of the stent. In particular, the grip member can be directly associated or integral with the core and can be adapted to releasably hold a self-expanding stent within the outer sleeve. Thus, the grip member may be a sleeve or coating attached around the periphery of the core, an integral portion of the core or a length of the core having a larger outer diameter than the remainder of the core. Particular embodiments include an instrument in which the grip member can be a sleeve of material with a friction contact surface or a sleeve of material that will take a set, for example a silicone rubber or polyurethane. In each of these embodiments the sleeve material may have an outer surface which can be substantially smooth and unbroken or an outer surface which can be roughened or irregular. Alternatively, where the core itself is made from a material that will take a set the grip member may be an integral portion of the core. An advantage of this embodiment can be that the grip member need not be a separate element which has to be attached or bonded to the core.

As used herein the term “friction” or “high friction” as applied to a material or its surface is intended to mean a material having a high coefficient of friction, i.e., a material whose surface offers high resistance to sliding motion; and the term “low friction” is intended to mean a material with a surface which offers little resistance to sliding motion and is relatively slippery.

Accordingly, since an important characteristic of the grip member can be that it should be capable of gripping or holding a stent and this capability can be effective while the stent can be retained within the instrument so that there can be no slippage when the core is moved forward or backward relative to the outer sleeve, it may be necessary when the grip member is a sleeve of material, that the material has a surface which offers high resistance to sliding motion. When the material is one which already has a high coefficient of friction the surface thereof that is in contact with the stent may be substantially smooth and unbroken. However, to increase the friction or enhance the inherent friction, the outer surface of the grip member may be roughened or irregular. In an alternative embodiment of the invention the gripping characteristic of the grip member may be achieved when the grip member comprises a coating of a releasable adhesive. In this embodiment the adhesiveness of the coating must be sufficient to retain or grip the stent without slipping while it is still within the instrument but weak enough to allow the stent to be released, by its own expansion, when it is free from the constraint of the outer sleeve. In a further embodiment of the invention the core itself can be made from a high friction material, for example, a polyurethane, and the grip member comprises a length of the core, at or near the distal end of the core, having a larger outer diameter than the remainder of the core. In practice, the enlargement of diameter may be relatively small, of the order of about 0.01 inch, but it has been found that mounting the stent on this thicker portion of the core provides sufficient grip to enable the instrument to be operated as desired.

When the core itself is made from a material that will take a set, such a material being inherently of high friction, the larger diameter may not be necessary for the formation of the grip member. A “material that will take a set” is defined herein as a material that will be locally deformed in situ by the compression of the stent when it is pressed against the core by the outer sleeve and will retain the deformation so that the stent is effectively gripped thereby. In each of the above-described embodiments the grip member may be at least as long as the stent. When an expandable stent to be deployed by an instrument according to the invention is made from a plurality of cross threads, for example, plastic or metal filaments, and particularly metal filaments, such as by the braiding operation described above, the ends of the stent will have a number of exposed filament ends. To avoid snagging of these exposed ends, for example, into the wall of the outer sleeve, and to avoid consequential damage either to the outer sleeve or to the stent itself, it may be advantageous to provide circumferential gaps adjacent the distal end and proximal end of the grip member to accommodate the respective ends of the stent. In this embodiment the ends of the stent tuck into the gaps thereby protecting them and preventing exposed filaments from snagging.

In contrast to the high friction characteristic of the grip member it may be desirable for the inner wall of the outer sleeve to have a low coefficient of friction to provide slidability and ease of movement of the stent-bearing grip member within the instrument. To achieve this characteristic the outer sleeve can be a hollow catheter made from a low friction material, for example, a fluorocarbon polymer such as polytetrafluoroethylene. Furthermore, to avoid the snagging problem mentioned above, it may be advantageous to provide the inner surface of the outer sleeve with a layer of hardened material. Such material also should be a low friction material. Additionally, when the outer sleeve is made from a relatively soft material, the soft distal end thereof may have a protective hard hollow cap, for example, made of metal, attached thereto.

An embodiment of the invention illustrated in FIG. 12 comprises an outer sleeve 81 having an integral handle 82 at its proximal end. The distal end 83 of the outer sleeve can be positioned within a body canal 84. Disposed axially within the outer sleeve can be a hollow core 85 having a handle 86 at its proximal end. The distal end 87 of the core has a stepped up diameter where it meets the distal end of the outer sleeve so that it provides a smooth transition from the end of the outer sleeve, and is also within the body canal. A guide wire 88 passes axially through the hollow core. Attached around the periphery of the core at its distal end can be a grip member 89 which releasably grips a self-expanding stent 90, shown here partly deployed at the proper location within the body canal. The stent may comprise materials and compositions as discussed above or below.

FIG. 13 is an enlarged side elevation showing a braided self-expanding stent 90, partly deployed from the distal end of the outer sleeve 91. This view shows the exposed ends of the wire filaments 91 which make up the stent. FIGS. 14 and 15 illustrate a grip member 89 made from a high friction material attached around the periphery of a hollow core 85. The grip member has circumferential gaps 92, 93 adjacent its distal end and proximal end, respectively, which accommodate the ends 91 of the stent, thereby avoiding snagging into the inner wall of the outer sleeve. FIG. 16 illustrates a grip member 89A which comprises a coating of releasable adhesive around the periphery of the inner core 85.

FIG. 17 illustrates, in cross-section, an embodiment in which the hollow core 85, can be made from a high friction material, for example, a polyurethane, and the grip member 89B comprises a length of the core having a larger diameter than the remainder of the core, indicated schematically by the step 94. If the high friction material is a material that will take a set the grip member may be simply a portion of the core without the enlargement of diameter. FIG. 18 is a side elevation, partly in section, of part of an embodiment showing additional features. In this embodiment the outer sleeve 81, which can be made from polytetrafluoroethylene, has a smooth metal rim 95 at its distal end to prevent snagging from the ends of the stent 90. The distal end 96 of the metal rim can be rounded to facilitate passage along the body canal. Additionally, the metal cap may serve as a marker element for fluoroscopic monitoring of the placement of the instrument within the body canal. Additional or alternative marker elements 97 may be provided at pre-determined positions on the outer sleeve and/or on the core (not shown). A flexible filiform 98 may be attached to the distal end of the core 85 to facilitate passage of the instrument along a body canal in a known manner. Deployment of a stent within a body canal in accordance with the method of the invention may be performed by using any of the embodiments illustrated in the drawings and described above.

A self-expanding stent can be introduced into the instrument in a manner known in the art and pre-located on the grip member. The grip member bearing the stent can be withdrawn into the instrument so that the whole of the stent can be within the outer sleeve, close to the distal end thereof, and can be constrained by the outer sleeve. The instrument containing the stent can be then introduced into the body canal, with or without the aid of a guide wire, and passed into the canal until it reaches a position for proper placement of the stent. The introduction and passage of the instrument in the body canal may be facilitated when a filiform is attached to the distal end of the core as described hereinabove. The positioning of the instrument within the body canal may be monitored and verified by any means known in the art, for example, by use of an endoscope, or by fluoroscopy or any other imaging technique.

When the correct position for proper placement of the stent is reached and verified, the stent can then be deployed by moving the outer sleeve relative to the core. This operation can be performed by holding the handle at the proximal end of the core so that the core, together with the grip member holding the stent, remains stationary, gripping the handle at the proximal end of the outer sleeve and withdrawing the latter towards the core handle so that the outer sleeve moves backward, thus exposing the stent, which, free from the constraint of the outer sleeve, expands to its expanded state. Before the stent is completely deployed from the instrument, the positioning thereof in the body canal can be checked. If the position is correct then the withdrawal of the outer sleeve can be continued until the stent is clear of the instrument and the instrument is then withdrawn from the body canal. However, if the monitoring reveals that the stent is not in its proper position then it may be retracted back within the outer sleeve simply by moving the core backwardly relative to outer sleeve using the handles on the core and outer sleeve. The instrument, containing the retracted stent, then may be re-positioned as required and the deployment operation repeated with the stent in its correct position.

Deployment of the stent by withdrawing the outer sleeve relative to the core has the advantage that it avoids the problem of the distal end of the stent digging into or snagging against the wall of the body canal, which problem might occur if the stent were to be pushed into the body canal from behind.

Another embodiment of the self-expanding prostheses (e.g., stent) comprises a flexible tubular body that can be composed of several individual rigid but flexible thread elements having spring properties. Each thread element extends in coil configuration with the center line of the body as a common axis, a number of elements having the same direction of winding but axially displaced relative to each other crossing a number of elements also axially displaced to each other but having the opposite direction of winding. These elements suitably form a braided configuration which by means of suitable members can be implanted in a radially contracted condition in for example blood vessels, urinary tracts, bilious tracts, gorges, or other positions that are difficult to access so that it after self-expansion will be fixed at the implantation site thus providing permanent support for the surrounding walls of the vessel.

The crossing thread elements can be symmetrically arranged in the form of a braid. If the known cell-loaded prosthesis is to be used for example for widening a narrow section in a blood vessel the flexible tubular body is suitably inserted arranged in a radially contracted state at the tip of a flexible instrument, for example percutaneously in a blood vessel. The device can then be intraluminally or transluminally transferred to the relevant narrow section of the blood vessel, and the tubular body can then be allowed to expand and, once in a radially expanded state, it can be released from the instrument so that it remains at the implantation site under self-fixation and the instrument can be removed. In this connection the diameter of the body in an unloaded and expanded state should be chosen somewhat larger than the inner diameter of the vessel. This results in a certain permanent pressure or engagement against the inner wall of the vessel which pressure has to be sufficiently large to keep the previous restriction open at the same time as an effective self-fixation will be obtained.

The thread elements can be built up as monofilaments, i.e., they can consist of single thread elements (e.g., resorbable plastic). Monofilaments have the advantage of being smooth on their external surface such that they can glide smoothly past each other.

A cell-loaded prosthesis of the type described above can be suitably manufactured starting from a tubular braid manufactured in a braiding machine known per se wherein usually a number of bobbins, each one containing its thread element, are movably arranged in a ring about a center, so that each bobbin can rotate about its own axis in connection with dewinding the respective thread elements, at the same time as the bobbins are moved about in a zig-zag-shaped circular movement about this center. A number of bobbins are arranged in the same manner in a ring but are moved in a zig-zag-shaped circular movement in the opposite direction in relation to the first-mentioned group of bobbins. The braid can be suitably deposited around a tubular axis in the center of the machine, and the thread elements can form different braiding patterns, e.g., depending on how the bobbins are brought to rotate. Tubular cell-loaded prosthesis of a suitable length can then be severed from the manufactured tubular braid.

In certain implementations the thread elements forming the tubular body may have a dimension which can be as small as possible but which at the same time provides for the necessary force against the wall of the vessel so that the tubular body obtains a small wall thickness so as not to accommodate too much space when for example implanted in fine blood vessels so that a too large reduction of the flow area for the blood will result. This can be particularly important with prostheses of a relatively small diameter, for example for use for implantation into the coronary vessels of the heart. Moreover, a small dimension of the thread elements can be essential in those cases where one wishes to obtain a high expansion capacity of the cell-loaded prosthesis, i.e., a high ratio between the cell-loaded prosthesis in an expanded state in relation to the cell-loaded prosthesis in a radially contracted state. Another reason as to why small dimensions of the thread elements may be desirable can be the fact that the cell-loaded prosthesis in a contracted state shall be accommodable in an implantation device of small diameter, for example for percutaneous implantation. Finally, small thickness of the thread elements can be an important advantage from a biological point of view, since the cell-loaded prosthesis built up from fine thread elements in an implanted state substantially facilitates coverage of the cell-loaded prosthesis with a layer of natural cells which in a blood vessel prevents the risk for thrombosis. For this reason it has been found that the thread elements must be made of a flexible, rigid, resilient material, for example a spring steel, a spring alloy or the like, the rigidity of the material in combination with its spring properties being of an essential importance. However, it may be found that it is in practice coupled with great difficulties to make a self-expanding cell-loaded prosthesis starting from a material of such properties.

Provided in one implementation is an elastic, self-expanding cell-loaded prosthesis, the supporting construction of which includes a flexible tubular body which can be composed of a plurality of individual rigid but elastically flexible thread elements having spring properties. The tubular body can be designed such that the remaining tension of the thread elements in the state in which they constitute supporting elements in the tubular body, at least at the end sections of the body are adjusted so that the diameter of an unloaded helix-shaped thread element, at least at its end sections, when removed from the other elements forming the tubular body is not more that about 60% larger than the diameter of the body in an unloaded condition. By the diameter of the thread element there is meant in the present context the diameter of the cylinder within which the helix-shaped thread element is considered to be inscribed.

In one embodiment at least one of the thread elements at each crossing site can be deformed in such a manner that it at least partly encloses the other thread element. When using thread elements of circular cross section the expression “at least partly circumscribes” but instead of point contact between crossing thread elements line contact would be obtained. The deformation of the outer thread element at each crossing site can be constituted by a breaking over the inner thread element at the area of contact between the two elements. This means in other words that each thread element of the tubular body extends alternatingly radially inside and radially outside the crossing thread elements at the respective crossing points, the number of thread elements in one rotational direction being the same as the number of thread elements in the other rotational direction. In the following this configuration will be called “one above/one below”.

The deformation of at least the outer thread element at each crossing site as described in the above embodiment results in the important advantage that relative sliding movement between the thread elements will be prevented or at any rate made quite difficult, and this in turn means that the solution of the above-indicated problem of the outward bending of the thread ends will be further facilitated. Alternative deformation techniques are conceivable, and another example is one where both thread elements at each crossing site are deformed in the opposite direction relative to each other. The deformation may also be constituted by flattening of the juxtaposed surfaces of crossing thread elements at the crossing site. It has also been found that by using the above-mentioned deformation there may also be gained the advantage that the disturbing tension of the thread elements can be reduced so that if a thread element is removed from the cell-loaded prosthesis it has a helix shape of largely the same pitch as when it was part of the cell-loaded prosthesis, i.e., by the deformation one can also remove a great part of the tensions.

The thread elements may all comprise biologically degradable material or alternatively only some of the threads may comprise biologically degradable material to provide more or less rigidity, and to allow for various mechanical aspects.

According to another aspect of the invention it has been found that in certain cases, particularly in prostheses of small diameter and built up of fine thread elements, it may be preferred to design the body so that at its ends in an unloaded condition it widens conically outwardly to a diameter which is greater than the diameter of the rest of the body. The conical widening outwardly can suitably be to a diameter which is at most about 20% greater than the diameter of the body in the intermediate section. The reason why this conical widening of the end sections of the cell-loaded prosthesis results in substantial advantages is the fact that in practice it has been found that the ends of the cell-loaded prosthesis at radial compression of same are subjected to a greater reduction of the diameter than the rest of the body. Since the cell-loaded prosthesis is intended to be implanted in a vessel of somewhat smaller diameter than the cell-loaded prosthesis has in an unloaded state the cell-loaded prosthesis when implanted will therefore obtain a substantially constant diameter across its full length. The desired conicity at the ends can suitably be obtained by adjusting the remaining tension of the thread elements or a selected deformation at the crossing sites.

In order to obtain a cell-loaded prosthesis with filtering function it may optionally be suitable to design at least one end of the body with a diminishing diameter, whereby it can serve as a filter when applied. According to yet another aspect of the invention the cell-loaded prosthesis can comprise extra threads of other materials in order that the cell-loaded prosthesis shall obtain the desired porosity. It may in this case also function as a graft.

One embodiment of such cell-loaded prosthesis according to the invention resides in the feature that in connection with the braiding operation in a conventional braiding machine known per se the tubular body can be braided under application of such tension to each individual thread element that they are permanently deformed and bent over the under-lying thread element at the crossing point. By applying this technique there will be obtained better adaptation of the remaining tension of the thread elements and also better fixation by bending of the crossing thread elements in relation to each other while maintaining flexibility of the cell-loaded prosthesis.

An alternative process according to the invention to provide for deformation of the thread elements in connection with the crossing points is to subject the body after its manufacture to mechanical deformation, so that at least one of the thread elements at each crossing point at least partly circumscribes the other thread element, so that sliding movement between the crossing threads can be prevented, whereas rotational movement under low friction between the thread elements at the crossing points will be made possible. Such mechanical deformation can be provided for example by hammering, mechanical or isostatic pressing or blastring. The mechanical deformation obviously mainly results in deformation of the outer thread element at each crossing point so that it at least partially will circumscribe the underlying thread element.

Another configuration would be the construction of a stent made of serially arranged segments of the design as outlined above, with the exception that each individual segment can be linked by a less rigidly constructed weave, which will permit sufficient bending to enable the stent to negotiate a tortuous structure. The linkage weave linking each segment could be an extension or component of the individual segments.

As previously indicated it may be desirable in certain embodiments to use as material for the thread elements materials, which are medicinally acceptable, are rigid and have adequate or extreme spring properties. Since it may be desirable that the wall of the cell-loaded prosthesis be as thin as possible and exert a certain sufficient pressure against the wall of the vessel at the same time as the cell-loaded prosthesis shall have a high expansion number, it may be preferred in accordance with these certain embodiments that the thread material should have such high “springiness” or energy storage capacity as possible in view of the other design parameters.

A biologically degradable cell-loaded prosthesis or stent may be positioned by means of compression and simultaneous axial extension over the tip of a small flexible implantation instrument provided with a central channel to enable i.e., insertion of a so called guide wire in the channel to facilitate insertion of the cell-loaded prosthesis. The cell-loaded prosthesis can be placed in a compressed state, surrounded by a thin plastic tube belonging to the tip of the instrument.

In another embodiment, illustrated in FIGS. 19-26, the biologically degradable stent comprises an open mesh or weave construction, consisting of two sets of oppositely directed, parallel and spaced apart helically wound strands or filaments indicated at 118 and 120, respectively. The stent comprises an elongate cylindrical core substantially uniform in lateral cross-section and having a core diameter, and an elongate tubular case or shell substantially uniform in lateral cross-section and having a case inside diameter, wherein one or both of the core and case can be formed of a biologically degradable material. In another embodiment, one of the core and case can be formed of a biologically degradable material and the other can be formed of a resilient material having a yield strength (0.2% offset) of at least 150,000 pounds per square inch (psi), wherein the core diameter can be less than the interior diameter of the case, and the lateral cross-sectional area of the core and case can be at most ten times the lateral cross-sectional area of the core; the core can be inserted into the case to form an elongate composite filament in which the case surrounds the core.

The sets of strands are interwoven in an over and under braided configuration so as to form multiple intersections, one of which is indicated at 122. The self-expanding stent 116 is illustrated in its relaxed state, where no external stress is applied. The filaments or strands of the stent 116 are resilient, permitting a radial compression of the stent into a reduced-radius, extended-length configuration suitable for intraluminal or transluminal delivery of the stent to the intended placement site. By selectively controlling the angle between the oppositely directed helical strands the degree of axial elongation for a given radial compression may be predetermined. According to the present invention, strands 118 and 120 of cell-loaded prosthesis 116 can comprise biologically degradable material either in those strands alone or multiple strands. As seen in FIGS. 21 and 22, a filament 118a of the cell-loaded prosthesis can be of composite construction, with a core 124 surrounded by and concentric with an annular resilient case 126.

Further, it can be advantageous in accordance with one aspect of the invention to form a cell-loaded prosthesis with substantial open space to promote embedding of the stent into tissue, and fibrotic growth through the stent wall to enhance long-term fixation. A more open construction also enables substantial radial compression of the cell-loaded prosthesis for deployment. In a typical construction suitable for intraluminal or transluminal implantation, the filaments can have a diameter of about 0.1 millimeter (0.004 inches), with adjacent parallel filaments spaced apart from one another by about 1-2 millimeters (0.04-0.08 inches) when the stent is in the relaxed state.

The process can begin with insertion of a solid cylinder or wire 128 of the core material into a central opening 130 of a tube 132 of the case material. Core wire 128 and tubing 132 are substantially uniform in transverse or lateral sections, i.e., sections taken perpendicular to the longitudinal or axial dimension. In general, the wire outer diameter can be sufficiently close to the tubing inner diameter to insure that core or wire 128, upon being inserted into opening 130, can be substantially radially centered within the tubing. At the same time, the interior tubing diameter must exceed the core outside diameter sufficiently to facilitate insertion of the wire into an extended length of wire and tubing, e.g., at least twenty feet.

Insertion of the core into the tube forms a composite filament 134. More particularly, in the illustrated example composite filament 134 can be drawn through three dies, indicated at 136, 138, and 140, respectively, as seen in FIG. 24. In each of the dies, composite filament 134 can be cold-worked in radial compression, causing the case tube 132 and the tantalum core wire 128 to cold flow in a manner that elongates the filament while reducing its diameter. Initially, case tube 132 can be elongated and radially reduced to a greater extent than core wire 128, due to the minute radial gap that allowed the insertion of the core into the tube. However, the radial gap can be closed rapidly as the filament is drawn through die 136, with subsequent pressure within die 136 and the remaining dies cold-working both the core and case together as if they were a single, solid filament. In fact, upon closure of the radial gap, the cold-working within all dies forms a pressure weld along the entire interface of the core and case, to form a bond between the core and case material.

As composite filament 134 is drawn through each die, the cold-working induces strain hardening and other stresses within the filament. Accordingly, one or more heating stages are provided, e.g., furnace 142. At each annealing stage, substantially all of the induced stresses are removed from the case and core, to permit further cold-working. Each annealing step can be accomplished in a brief time, e.g., in as few as one to fifteen seconds at annealing temperature, depending on the size of composite filament 134. While FIG. 24 illustrates one cold-working stage and annealing stage, it is to be understood that the appropriate number of stages can be selected in accordance with the desired final filament size, the desired, degree of cross-sectional area reduction during the final cold-working stage, and the initial filament size prior to cold-working.

In FIG. 26, several filaments or strands 134a-e are helically wound about a cylindrical form 148 and held in place at their opposite ends by sets of bobbins 150a-e and 152a-e. Strands 134a-e can be individually processed, or individual segments of a single annealed and cold-worked composite filament, cut after the final cold-working stage. In either event, the filaments cooperate to form one of the two oppositely directed sets of spaced apart and parallel filaments that form a device such as stent 116. While only one set of filaments are shown, it is to be understood that a corresponding group of filaments, helically wound and intertwined about form 148 in the opposite direction, are supported by corresponding bobbins at the opposite filament ends.

FIG. 27 illustrates two filaments 134a and 154a, one from each of the oppositely wound filament sets, supported by respective bobbins 150a/152a and 156a/158a. The filaments overlay one another to form several intersections, one of which is indicated at 162. When the filaments are properly tensioned, a slight impression can be formed in the overlying filament at each intersection. These impressions, or saddles, tend to positionally lock the filaments relative to one another at the intersections, maintaining the cell-loaded prosthesis configuration without the need for welding or other bonding of filaments at their intersections.

FIGS. 30 and 31 show a further alternative composite filament 180, consisting of a central core 182, an outer annular structural case 184, and an intermediate annular layer 186 between the core and the case. Intermediate layer 186 provides a barrier between the core and case, and can be particularly useful in composite filaments employing core and case materials that would be incompatible if contiguous, e.g., due to a tendency to form intermetallics. Materials suitable for barrier layer 186 may include tantalum, niobium, platinum, or combinations thereof. As suggested by FIG. 30, the core, barrier layer and case can be provided as a cylinder and two tubes, inserted into one another for manufacture of the composite filament as explained above.

FIG. 32 illustrates another alternative embodiment composite filament 188 having a central core 190, a structural case 192, and a relatively thin annular outer cover layer 194. Composite filament 188 can be particularly useful when the selected mechanical structure lacks satisfactory biocompatibility, hemocompatibility, or both. Suitable materials for cover layer 194 include tantalum, platinum, iridium, niobium, titanium, and stainless steel. The composite filament can be manufactured as explained above, beginning with insertion of the core into the structural case, and in turn, inserting the case into a tube formed of the cover material. Alternatively, cover layer 194 can be applied by a vacuum deposition process, as a thin layer (e.g., from ten to a few hundred microns) may be all that is required.

Resilient or self-expanding prostheses can in accordance with one aspect of the invention be deployed without dilation balloons or other stent expanding means. Self-expanding stents can be preselected according to the diameter of the blood vessel or other intended fixation site. Further, the self-expanding stent remains at least slightly elastically compressed after fixation, and thus has a restoring force which facilitates acute fixation.

FIG. 33 illustrates a cell-loaded stent or prosthesis in accordance with one embodiment.

The cell-loaded device of the present invention can be deployed to the site of vascular pathology and possess the following properties in connection with the treatment of vascular narrowing or out pouching. These properties include (1) the ability to be selectively expandable to occlude a narrow vascular segment, or to occlude the opening of an out pouch, (2) the ability to re-establish vessel wall anatomy, and (3) the construction with material which would permit radiographic detection without distortion of the surrounding anatomic relations. An intravascular stent comprising, for example, lactic acid polymers designed, for example, either to expand a narrow segment or to occlude the opening of an out pouch, is herein disclosed.

Such materials may take the form of a sheet or be composed of thin threads woven in such a manner, which will permit the stents to assume the desired form and size (i.e., with predetermined memory) upon deployment. Desirable features of the stents would be the ability of the stents to negotiate through multiple bends in the vascular tree without loss of the ability to manipulate them. This has been the major deficiency in current stent designs rendering them too stiff, and thus, usable primarily in situations where the vessels are relatively straight such as the cervial carotid arteries, the vertebral/basilar arteries or the coronary arteries.

The designs which would likely permit this, in addition or supplemental to those discussed above, include ones in which elements of the stents can be manipulated to move relative to each other. An example would be a sliding weave pattern in the thin threads which are so woven that the part of the stent on the inside of a bend would be bunched closer together while the part on the outside of the bend would stretch out. Another design would be a construction of the stent, which incorporates multiple cylindrical elements linked together in series such that parts of each cylindrical element could be manipulated to be closer (to accommodate the inside of a bend) or farther apart (to accommodate the outside of a bend). In this configuration, the intervening portions of the stent linking the cylindrical elements together would form a smooth surface not to create any turbulent blood flow which in turn would lead to clot formation and occlusion of the vessel.

Such stents can be deployed using currently available deployment mechanisms such as, in addition or supplemental to those discussed above, the ensheathment in a catheter, which can be brought to the site of deployment. Upon correct positioning of the stent, it can be pushed out to the vascular lumen where it expands in a preformed manner to distend the vessel and/or to occlude any out pouching. Another method of deployment would be the delivery of the stent to the site on the external aspect of a balloon of a balloon catheter. Upon correct positioning of the balloon, it can be then inflated to the desired size thus forcing the stent onto or against the vascular wall. The stent can be then detached in situ. In embodiments wherein the stent is so constructed with memory to assume a preformed shape and size, it will maintain the vessel open in the desired configuration.

Stents which can be molded intravascularly to conform to the internal anatomy of the vessel in accordance with an aspect of the invention are composed of materials, which can be molded to a desired shape and size inside a vessel to conform to its anatomy. The material must also be flexible enough to allow the stent to be manipulated through bends in the vascular tree. One such material would be the amino acid polymers which can be formed to assume a certain shape and size, and which upon heating, can be molded into any form or size within defined constrains.

Stents of this type can be delivered to the vascular site on the external surface of a balloon catheter. In this stent design, the stent can be configured to assume the shape of a cylinder of different length or a sequence of spirals so that it can be secured onto the external surface of the catheter/balloon, which will then deliver it to the intended vascular site. These stents should be composed of material to allow them to be expanded reversibly to form a cylinder of larger diameter either through its inherent design (e.g., a series of spirals) or under specific conditions such as heating to a defined temperature. Once the stent is in the expanded state, and maintained in this state by the inflated balloon, it can be maintained in this expanded state by itself because of its inherent design or through termination of the expanding condition (e.g., termination of heating). The moment it is deemed that the stent can be correctly deployed (i.e., to be in the correct position and in the correct shape and size), it can be detached from the balloon by deflating the balloon. The deflated balloon can then be removed from the vessel with the catheter. There should also be memory in the initial configuration of the stent such that should the deployment of the stent and its expanded shape and size not be ideal, removal of the configuration condition such as the termination of heating would allow it to shrink back to its original smaller size and shape. In so doing, it can be resecured onto the external surface of the catheter and/or balloon to be redeployed.

The deployment balloon catheter can be composed of a catheter at the tip of which can be an inflatable balloon of defined length and expandable diameter. Impregnated in the catheter as well as the balloon surface are heating elements. These heating elements in the balloon wall can be so configured to be expandable as the balloon is inflated and yet be able to deliver a consistent and uniform degree of heating throughout the surface of the balloon, which is in contact with the stent. There may also be elements on the external surface of the catheter and/or balloon, which will secure the stent onto its surface such that the stent will not be detachable until it is positioned in the desired location inside the blood vessel.

Stents of this design are delivered to the vascular site on the external surface of the balloon. It can be maintained in a flexible state on account of its construction (e.g., a series of spirals) or under constant heating through the heating elements on the surface of the balloon so that it can negotiate the bends of the vascular tree. Upon arrival at the desired vascular site, the balloon can be inflated to expand the stent to the desired size and shape. The stent can be expanded because of its inherent design (e.g., a series of spirals), or through heating with the heating elements in the wall of the balloon. In the situation of a narrowed artery, inflation of the balloon can be used not only to expand the stent but also to dilate the vessel in turn. In the situation of the arterial out pouching, the stent will be expanded to conform to the internal configuration of the blood vessel but in such a position to occlude the opening of the out pouching. Once the stent is in place on the inner surface of the blood vessel, either its specific design (e.g., a series of spirals) would maintain it in place in the expanded state, or it can be maintained in the expanded state by termination of the condition, which allows it to be expanded. In the latter situation, heating allows the balloon to expand the stent to the desired shape and size. Cessation of heating would allow the stent to maintain its expanded shape and size on the inner surface of the vessel wall. After it is determined that the stent is correctly deployed, the balloon can be then deflated and removed from the circulation. The stent can be left at the vascular site either to maintain the vessel in its more dilated state or to close the opening of the out pouching on the side of the vessel.

Should the initial deployment not be ideal and that the stent needs to be repositioned, the design (e.g., a series of spirals) which permits it to be maintained in the expanded state should also permit it to be reversibly returned to the non-expanded state so that it can be retrieved and secured onto the surface of the balloon to be redeployed. If the design is such that the stent is composed of material to allow it to be expanded reversibly to form a cylinder of larger diameter under specific conditions such as heating to a defined temperature, the stent material should possess such memory such that the stent can be returned to its origin shape and size by removal of the configuration condition such as the termination of heating and deflation of the balloon. This would allow it to shrink back to its original smaller size and shape. In so doing, it can be resecured onto the external surface of the catheter and/or balloon to be redeployed.

FIGS. 34-37 illustrate a resorbable stent or cell-loaded prosthesis (“resorbable cell-loaded prosthesis”) in accordance with one aspect of the present invention. In accordance with one aspect of the invention, the stent or cell-loaded prosthesis comprises any non-metallic (e.g., polymer or plastic) material. In the illustrated embodiment, the stent or cell-loaded prosthesis comprises a resorbable material. The resorbable cell-loaded prosthesis can be either injection molded or machined to have an original shape (i.e., a natural shape that the material has before being heated to its glass transition temperature and deformed). The original shape can approximate the outer surface of the balloon, so that the resorbable cell-loaded prosthesis can be closely formed around the outer surface of the balloon. Alternatively, the resorbable cell-loaded prosthesis may be formed to have an original shape which can be different than that of the outer surface of the balloon, and the resorbable cell-loaded prosthesis may be heated to its glass transition temperature and formed relatively closely around the balloon. For example, the resorbable cell-loaded prosthesis may be formed to have a cylindrical shape and a diameter less than the maximum diameter of the balloon, and, subsequently, the resorbable cell-loaded prosthesis can be placed around the balloon. The resorbable cell-loaded prosthesis can then be brought to its glass transition temperature and formed around the balloon as the balloon slightly stretches (i.e., increases the diameter of) the resorbable cell-loaded prosthesis at glass transition temperature. The resorbable cell-loaded prosthesis is then cooled with the balloon remaining in the same shape so that the resorbable cell-loaded prosthesis fits snugly around the balloon, thus yielding the configuration of FIG. 34.

The balloon catheter can comprises conductors for transferring energy to the resorbable cell-loaded prosthesis to thereby bring the resorbable cell-loaded prosthesis to its glass transition temperature. The conductors can be distributed on the surface of the balloon to transfer thermal energy to the resorbable cell-loaded prosthesis. A control may be provided for selectively regulating the electrical energy used to heat the resorbable cell-loaded prosthesis. The expandable balloon may contain an inner wall lined with conductors which can be heated. The balloon may also be heated using a fluid that is heated to the glass transition temperature of the resorbable cell-loaded prosthesis.

FIG. 34 illustrates the resorbable cell-loaded prosthesis in a position coupled to a balloon. The balloon may comprise optional protrusions at the proximal and distal ends of the balloon in the form of discrete protrusions or proximal and distal rings. Additionally or alternatively, the resorbable cell-loaded prosthesis can be weaved in a manner in which the inside is relatively rough to create a frictional contact between the resorbable cell-loaded prosthesis and the balloon when the two are coupled together. Moreover, other attachment means which are known in the art may be used to secure the resorbable cell-loaded prosthesis.

In FIG. 35 the resorbable cell-loaded prosthesis is first heated by the balloon to its glass transition temperature. Subsequently, the balloon can be expanded to thereby expand the resorbable cell-loaded prosthesis as shown in FIGS. 35 and 36. Any conventional means of expanding the balloon may be used. As presently embodied, fluid can be passed through the center of the balloon, expanding the heated balloon. The fluid may comprise, for example, a saline solution or air. The resorbable cell-loaded prosthesis, which is heated to its glass transition state, correspondingly increases in diameter as the balloon expands. The balloon can be configured to expand the resorbable cell-loaded prosthesis into a cylindrical shape to match the inner wall of the vessel. In other embodiments, the balloon can be configured to form the resorbable cell-loaded prosthesis into other shapes.

The balloon's surfaces will press against the resorbable cell-loaded prosthesis, reforming the resorbable cell-loaded prosthesis so that it is no longer concave similar to the resorbable cell-loaded prosthesis in FIG. 35. Thus, once the temperature decreases and the balloon begins to deflate the now cylindrically-shaped resorbable cell-loaded prosthesis will no longer be affixed to the balloon, and will remain in its expanded configuration, allowing for removal of the balloon catheter.

FIG. 36 portrays the resorbable cell-loaded prosthesis at its maximally expanded state, at which time energy is no longer transferred to the resorbable cell-loaded prosthesis so that the resorbable cell-loaded prosthesis may cool below its glass transition temperature to body temperature, with surrounding liquids in the vessel rapidly absorbing much of the energy from the resorbable cell-loaded prosthesis as the resorbable cell-loaded prosthesis cools.

The balloon can then be deflated and removed from within the resorbable cell-loaded prosthesis as portrayed in FIG. 37. The resorbable cell-loaded prosthesis will hold this shape until its temperature is once again raised to the glass transition temperature.

The balloon, in one embodiment, can be re-inserted and expanded to contact the resorbable cell-loaded prosthesis. The balloon can then be energized to heat the resorbable cell-loaded prosthesis to its glass transition temperature, making the resorbable cell-loaded prosthesis malleable. The balloon can then be then slowly deflated, as heat is applied to the resorbable cell-loaded prosthesis, so that the resorbable cell-loaded prosthesis remains in its glass transition state and slowly moves back to its original shape. The resorbable cell-loaded prosthesis will return to its original small diameter shape as the balloon deflates more and more. The decreased size of the stent then allows for removal of the stent.

The resorbable cell-loaded prosthesis may alternatively be inserted with, for example, positioning and heating devices as are described in U.S. Pat. No. 3,868,956, the entire contents of which are herein incorporated by reference.

A stent or cell-loaded prosthesis may, alternatively, comprise materials in the form of a sheet composed of thin biologically degradable threads woven in such a manner, which will permit the stent to assume the desired form and size (i.e., with the predetermined memory) upon deployment. The stent may take the form of a typical self-expanding stent as disclosed in U.S. Pat. No. 4,655,771 which is incorporated herein by reference. Such a stent can be a radially and axially flexible, elastic tubular body with a predetermined diameter that can be variable under axial movement of ends of the body relative to each other and which can be composed of a plurality of individually rigid but flexible and elastic thread elements defining a radially self-expanding helix. This type of stent is known in the art as a braided stent.

Placement of any of the above-described stents, to the extent practicable, may be inserted using the structures and methods of U.S. Pat. No. 4,665,771. The stent is inserted in a body vessel in one embodiment by a device which comprises the use of a piston or, in another embodiment, by use of latch means to push the stent forward.

Any of the above-described stents, to the extent practicable, may be inserted using the structures and methods of U.S. Pat. No. 4,768,507. U.S. Pat. No. 4,768,507 discloses a stent insertion apparatus which includes an inner core member with a spiral groove formed on its outer surface, which groove cooperates with an outer sheathing to form a spiral cavity adapted to contain an expandable coil stent. The coil stent can be held in a radially compressed state within the spiral cavity by exerting a radial outward force on the outer sheath. The outer surface of the inner core member can be slidably mounted within the hollow outer sheath cylinder so that the spiral cavity is adapted to contain only the coil stent for which it is designed.

U.S. Pat. No. 4,743,152 discloses a device for implantation of a substantially tubular, radially expandable cell-loaded prosthesis including in combination the radially expandable cell-loaded prosthesis surrounding and concentric with a flexible probe and means for maintaining the cell-loaded prosthesis in a radially contracted state and for releasing the expandable cell-loaded prosthesis, wherein the means for maintaining and releasing the cell-loaded prosthesis comprises a hose concentrically surrounding the probe with one end of the hose being connected to the probe, the hose being folded inside itself, a double-walled section of the hose formed by the hose being folded inside itself, the double-walled section radially surrounding the cell-loaded prosthesis, a fluid-tight chamber provided between and defined by the probe and the hose, means for introducing and pressurizing a fluid in the chamber to reduce contact pressure and friction between the double-walled section of the hose, the cell-loaded prosthesis being released from the hose by axial relative movement of the one end of the hose with respect to an opposite end of the hose, the opposite end of the hose connected to an element of the device. The technology of this patent, to the extent practicable and compatible, can be used with any of the above-described stents and prostheses.

4. Addition of Regenerative Cells to Cell Carrier Portion of Prosthesis

The cell carrier can be appropriately seeded with regenerative cells to facilitate or optimize tissue generation or treatment in the intended area. The cell carrier may be a solid, resorbable or non-resorbable polymer based scaffold with a fiber or sponge macrostructure into which cells are seeded, or a hydrogel, e.g., fibrin, polyethylene glycol, in which cells are encapsulated. The carrier may be modified by a chemical, peptide, protein, or gene to support cell attachment, e.g., RGD, cell differentiation, e.g., CBFA-1gene, calcification, e.g., phosphor group. The cells can either be seeded in vivo by injection or other appropriate method at the treatment site, or seeded ex vivo.

Ex vivo seeding methods for solid based scaffolds encompassed in this invention can include art-recognized methods such as static seeding (or capillary action), injection, dynamic seeding, including uni- and bidirectional perfusion, spinner flask, orbital or random shaker, rotating or end-over-end bioreactor seeding, and vacuum assisted seeding, or a mixture of the methods, to name a few. Seeding methods for natural or synthetic gel based carrier encompassed in this invention may include art-recognized methods such as mixing the cells with the liquid components of the gel based carrier and forming a gel by physical interaction crosslinks (e.g. hydrogen, van der waals forces, polar forces, ionic bonds) or covalent crosslinks. Seeding using gel based carriers can be accomplished either in vivo or ex vivo. If the cell carrier portion is of a particulate form, the seeding methods may be as described above, or may be different. For example, the cells may simply be mixed with the cell carrier portion and placed, press fit or injected the cell/particulate mixture into the intended area of treatment, such as around the prosthesis.

The cells to be incorporated into the carrier may be previously incubated or cultured with any substance that may promote the cells' ability to adhere to the carrier or to stimulate the cells' tissue treating or forming capacity, the latter being exemplified by growth promoting proteins or genes, or other growth regulatory molecules. In an ex vivo seeding approach, the cell laden cell carriers can then be placed into the intended area of tissue treatment or formation to induce a desired response. Once implanted, host cell influx and ADC proliferation, differentiation, regenerative cell deposition, and, in cases of resorbable carriers, concomitant reabsorption, proceeds.

These and other methods of seeding the cell carriers may be disclosed in whole or in part, for example, in one or more of S. L. Ishaug, G. M. Crane, M. J. Miller, A. W. Yasko, M. J. Yaszemski, and A. G. Mikos. Bone formation by three-dimensional stromal osteoblast culture in biodegradable polymer scaffolds. J Biomed Mater Res 36 (1):17-28, 1997; H. L. Wald, G. Sarakinos, M. D. Lyman, A. G. Mikos, J. P. Vacanti, and R. Langer. Cell seeding in porous transplantation devices. Biomaterials 14 (4):270-278, 1993; N. S. Dunkelman, M. P. Zimber, R. G. LeBaron, R. Pavelec, M. Kwan, and A. F. Purchio. Cartilage production by rabbit articular chondrocytes on polyglycolic acid scaffolds in a closed bioreactor system. Biotechnol Bioeng 46:299-305, 1995; L. E. Freed, A. P. Hollander, I. Martin, J. R. Barry, R. Langer, and G. Vunjak-Novakovic. Chondrogenesis in a cell-polymer-bioreactor system. Exp Cell Res 240 (1):58-65, 1998; R. E. Schreiber, N. S. Dunkelman, G. Naughton, and A. Ratcliffe. A method for tissue engineering of cartilage by cell seeding on bioresorbable scaffolds. Ann NY Acad Sci 875:398-404, 1999; G. Vunjak-Novakovic, et al (1999) van Wachem P B, tronck J W, oers-Zuideveld R, ijk F, and ildevuur C R. Vacuum cell seeding: a new method for the fast application of an evenly distributed cell layer on porous vascular grafts. Biomaterials 11 (8):602-606, 1990; L. E. Freed, J. C. Marquis, G. Vunjak-Novakovic, J. Emmanual, and R. Langer. Composition of cell-polymer cartilage implants. Biotechnol. Bioeng. 43:605-614, 1994; J. L. van Susante, P. Buma, G. J. van Osch, D. Versleyen, P. M. van der Kraan, W. B. van der Berg, and G. N. Homminga. Culture of chondrocytes in alginate and collagen carrier gels. Acta Orthop Scand 66 (6):549-56, 1995; J. Elisseeff, W. McIntosh, K. Anseth, S. Riley, P. Ragan, and R. Langer. Photoencapsulation of chondrocytes in poly(ethylene oxide)-based semi-interpenetrating networks. J. Biomed. Mater. Res. 51:164-171, 2000.

Once seeded on the cell carrier, the device can be inserted into an intended area of tissue treatment or formation in the recipient. Alternatively, the cells can be seeded onto the cell carrier subsequent to insertion of the prosthesis into an intended area of tissue treatment or formation in the recipient. Once the device is inserted, in-vivo cellular proliferation and, in the case of resorbable cell carriers, concomitant reabsorption of the cell carrier, may proceed.

In accordance with another aspect of the invention, the cell carrier portion may further comprise a substance (e.g., a chemical or biological molecule) to facilitate cell seeding, for example, an adhesion peptide or other biological molecule to enhance cell attachment, a substance that alters the hydrophilicity of the cell carrier, a surface charge, a polar molecule, or a surfactant or wetting agent to increase cell attachment or increase fluid uptake into the cell carrier. The substance used to facilitate cell seeding may be located on the surface of the cell carrier pores, the outer regions of the cell carrier, interspersed in the bulk of the cell carrier material, at predetermined locations, or any combinations thereof.

5. Use of the Cell-Loaded Prosthesis for Tissue Treatment or Formation

The cells may also be applied with additives to enhance, control, or otherwise direct the intended therapeutic effect. Cells may be administered following genetic manipulation such that they express gene products that are believed to or are intended to promote the therapeutic response(s) provided by the cells. Examples of manipulations include manipulations to control (increase or decrease) expression of factors promoting tissue formation, expression of developmental genes promoting differentiation into specific vessel or cardio lineages or that stimulate tissue growth and proliferation.

The active cell population may be applied to the resorbable cell carrier of the present device alone or in combination with other cells, tissue, tissue fragments, growth factors, or other additive intended to enhance the delivery, efficacy, tolerability, or function of the population. The cell population may also be modified by insertion of DNA in a plasmid or viral vector or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Muramatsu et al., 1998).

In another aspect, the cells could be combined with a gene encoding a growth factor(s) which would allow cells to act as their own source of growth factor during, for example, vessel or cardio. Genes encoding anti-apoptotic factors or agents can also be applied. Addition of the gene (or combination of genes) could be by any technology known in the art including but not limited to adenoviral transduction, “gene guns,” liposome-mediated transduction, and retrovirus or lentivirus-mediated transduction, plasmid, adeno-associated virus. Cells may be implanted along with a carrier material bearing gene delivery vehicle capable of releasing and/or presenting genes to the cells over time such that transduction can continue or be initiated in situ. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No. 20020182211. A preferred immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the ADC of the invention.

In certain embodiments of the invention, the cells are administered to a patient with one or more cellular differentiation agents, such as cytokines and growth factors. Examples of various cell differentiation agents are disclosed in (Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001).

A patient with a history and physical exam consistent with coronary vascular disease may be a candidate for therapy in accordance with an aspect of the present invention. A person skilled in the suitable field can perform a routine percutaneous transluminal coronary angioplasty (PTCA). The material can be removed and placed in a chamber which will seed the prosthesis to form, for example, a polymer coated stent with a cell population including but not limited to: adipose derived stem cell or, in modified embodiments, any other mesenchymal stem cell from other tissue sources including bone marrow, endothelial progenitor cells and mature endothelial cells. After seeding, the stent can be applied to the endolumen in a standard fashion. The patient may then recover and be monitored after the procedure using standard of care.

While embodiments of the present invention have been illustrated in detail, it will be apparent that modifications and adaptations of the embodiments will occur to those skilled in the art. However, it is to be expressly understood that such modifications and adaptations are within the spirit and scope of the present invention.

What is claimed is:

1. A tubular prosthesis for insertion into a body passage, the tubular body comprising a cell carrier portion which is compatible with living tissue and which is sized and shaped to conform to and expand a narrow segment of the body passage, wherein the cell carrier portion is further comprised of regenerative cells.

2. The tubular prosthesis as set forth in claim 1, wherein the cell carrier portion comprises polylactide.

3. The tubular prosthesis as set forth in claim 1, wherein the cell carrier portion comprises at least one of a poly-lactide polymer and a copolymer of two or more poly-lactides.

4. The tubular prosthesis as set forth in claim 1, wherein the cell carrier portion is resorbable.

5. The tubular prosthesis as set forth in claim 1, wherein the tubular body is partially resorbable.

6. The tubular prosthesis as set forth in claim 1, wherein the regenerative cells are stem cells.

7. The tubular prosthesis as set forth in claim 1, wherein the regenerative cells are progenitor cells.

8. The tubular prosthesis as set forth in claim 1, wherein the regenerative cells are endothelial progenitor cells.

9. The tubular prosthesis as set forth in claim 1, wherein the regenerative cells are a combination of stem cells and progenitor cells.

10. The tubular prosthesis as set forth in claim 9, wherein the regenerative cells are derived from adipose tissue.

11. The tubular prosthesis as set forth in claim 9, wherein the regenerative cells are bone marrow derived cells.

12. The tubular prosthesis as set forth in claim 9, wherein the regenerative cells are embryonic cells.

13. The tubular prosthesis as set forth in claim 9, wherein the regenerative cells are derived from blood.

14. The tubular prosthesis as set forth in claim 9, wherein the cell carrier portion is pre-loaded on the tubular prosthesis during manufacturing.

15. The tubular prosthesis as set forth in claim 14, wherein the regenerative cells are loaded to the tubular prosthesis after manufacturing and prior to insertion into the body passage.

16. The tubular prosthesis as set forth in claim 1, wherein the cell carrier portion and the regenerative cells are loaded simultaneously onto the tubular prosthesis prior to insertion into the body passage.

17. The tubular prosthesis as set forth in claim 1, wherein the body passage is within the cardiovascular system.

18. The tubular prosthesis as set forth in claim 1, wherein the regenerative cells regenerate the endothelial cells in and around the body passage.

19. The tubular prosthesis as set forth in claim 1, wherein the regenerative cells promote collateralogenesis in and around the body passage.

20. The tubular prosthesis as set forth in claim 1, wherein the regenerative cells promote cardiomyocytic differentiation in and around the body passage.
